(19) United States
(12) Patent Application Publication
(10) Pub. No.: US 2022/0026477 A1
(43) Pub. Date: Jan. 27, 2022

(54) PULSE PROCESSING DEVICE AND METHOD OF ASSOCIATING PULSE-RELATED WAVELET COEFFICIENTS TO A CORRESPONDING REFERENCE PULSE SHAPE

(71) Applicant: UNIVERSITÉ LAVAL, Quebec (CA)

(72) Inventors: Gabriel GAGNON-TURCOTTE, Québec (CA); Benoit GOSSELIN, Lac Beauport (CA)

(21) Appl. No.: 16/936,589

(22) Filed: Jul. 23, 2020

(51) Int. Cl. G01R 29/027; G01R 19/04; A61B 5/024; A61B 5/00

(52) U.S. Cl. CPC G01R 29/0273; A61B 5/4064; A61B 5/024; G01R 19/04

(57) ABSTRACT

There is described a method of associating a pulsed signal to a corresponding reference pulse shape. The method generally has accessing reference data having a plurality of reference pulse shapes, each reference pulse shape having a sparse array of average coefficients; receiving a pulsed signal having an array of amplitude values, including generating a sparse array of instantaneous coefficients based on said pulsed signal for example using a discrete wavelet transform; calculating a plurality of first distances between said instantaneous coefficients of said sparse array and the average coefficients of each one of said reference pulse shapes, said first distances having a first minimal distance identifying a closer one of the reference pulse shapes; and upon determining that said first minimal distance is below a first distance threshold, associating said sparse array of instantaneous coefficients to the closer one of the reference pulse shapes.

### FIELD

The improvements generally relate to devices for processing electrical pulses, and more specifically relate to standalone pulse processing apparatuses.

### BACKGROUND

Processing electrical signals such as neural signals or cardiac signals, including for instance the real-time or quasi real-time detection and classification thereof, generally require a considerable amount of hardware and software. In applications where a subject's brain tissues are to be interrogated, the heaviness of existing head-mounted pulse processing apparatuses may prevent normal behaviour, which may taint at least some in situ experiments. Although existing pulse processing apparatuses are satisfactory to a certain degree, there remains room for improvement, especially in reducing their footprint and/or power consumption.

### SUMMARY

In an aspect of the present disclosure, there is provided methods and devices for processing electrical pulses. The processing of the electrical pulses can require the detection of the electrical pulses, including the generation of arrays of amplitudes values indicative of the detected electrical pulses; the compression of the arrays of amplitudes values, including the dimension reduction of these arrays of amplitudes values resulting into arrays of wavelet coefficients; and the classification of the arrays of wavelet coefficients into respective reference pulse shapes.

In some embodiments, the compression of a received array of amplitudes values into a corresponding array of wavelet coefficients can be performed using a hardware coded sorting tree algorithm, which can reduce the amount of computational power and/or memory requirements on the pulse processing device compared to existing pulse processing apparatuses.

In accordance with a first aspect of the present disclosure, there is provided a method of associating a pulsed signal to a corresponding reference pulse shape, the method comprising: accessing reference data having a plurality of reference pulse shapes, each reference pulse shape having a sparse array of average coefficients; receiving a pulsed signal having an array of amplitude values, including generating a sparse array of instantaneous coefficients based on said pulsed signal; calculating a plurality of first distances between said instantaneous coefficients of said sparse array and the average coefficients of each one of said reference pulse shapes, said first distances having a first minimal distance identifying a closer one of the reference pulse shapes; and upon determining that said first minimal distance is below a first distance threshold, associating said sparse array of instantaneous coefficients to the closer one of the reference pulse shapes.

Further in accordance with the first aspect of the present disclosure, the method can for example register the sparse array of instantaneous coefficients as a sparse array of average coefficients of a new reference pulse shape in the reference data.

Still further in accordance with the first aspect of the present disclosure, at least one of said first distances can for example be a L1 distance given by a relation equivalent to the following equation:

d_{L1}=Σᵢ₌₁^{N}|xᵢ−x̅ᵢ̅|,

wherein i denotes an integer, N denotes a number of coefficients of said sparse arrays, xᵢ denotes the i^{th} instantaneous coefficients of said sparse array and x̅ᵢ̅ denotes the i^{th} average coefficients of a corresponding one of the reference pulse shapes.

Still further in accordance with the first aspect of the present disclosure, the method can for example further comprise updating said average coefficients of the closer one of the reference pulse shapes based on said instantaneous coefficients.

Still further in accordance with the first aspect of the present disclosure, the method can for example further comprise calculating a plurality of second distances between the updated average coefficients of the closer one of the reference pulse shapes and the average coefficients of the other ones of the reference pulse shapes, the second distances having a second minimal distance identifying a closer one of said other ones of the reference pulse shapes.

Still further in accordance with the first aspect of the present disclosure, the method can for example further comprise, upon determining that said second minimal distance is greater a second distance threshold, registering the instantaneous coefficients as a sparse array of average coefficients of a new reference pulse shape in the reference data.

Still further in accordance with the first aspect of the present disclosure, said generating said sparse array of instantaneous coefficients can for example include performing one or more discrete transforms on said amplitude values of said pulsed signal.

Still further in accordance with the first aspect of the present disclosure, said performing can for example include performing one or more discrete wavelet transforms on said amplitude values of said pulsed signal, said instantaneous and said average coefficients being wavelet coefficients.

Still further in accordance with the first aspect of the present disclosure, said discrete wavelet transform can for example be a Symmlet-2 discrete wavelet transform.

In accordance with a second aspect of the present disclosure, there is provided a pulse processing device comprising: a substrate; and a pulse classification module mounted on the substrate, the pulse classification module having a processor and a memory having instructions stored thereon that when performed by the processor perform the steps of: accessing reference data having a plurality of reference pulse shapes, each reference pulse shape having a sparse array of average coefficients; receiving a pulsed signal having an array of amplitude values, including generating a sparse array of instantaneous coefficients based on said pulsed signal; calculating a plurality of first distances between said instantaneous coefficients of said sparse array and the average coefficients of each one of said reference pulse shapes, said first distances having a first minimal distance identifying a closer one of the reference pulse shapes; and upon determining that said first minimal distance is below a first distance threshold, associating said sparse array of instantaneous coefficients to the closer one of the reference pulse shapes.

Further in accordance with the second aspect of the present disclosure, the pulse processing device can for example further comprise, upon determining that said first minimal distance exceeds said first distance threshold, registering the sparse array of instantaneous coefficients as a sparse array of average coefficients of a new reference pulse shape in the reference data.

Still further in accordance with the second aspect of the present disclosure, at least one of said first distances can for example be a L1 distance given by a relation equivalent to the following equation:

d_{L1}=Σᵢ₌₁^{N}|xᵢ−x̅ᵢ̅|,

wherein i denotes an integer, N denotes a number of coefficients of said sparse arrays, xᵢ denotes the i^{th} instantaneous coefficients of said sparse array and x̅ᵢ̅ denotes the i^{th} average coefficients of a corresponding one of the reference pulse shapes.

Still further in accordance with the second aspect of the present disclosure, the pulse processing device can for example further comprise updating said average coefficients of the closer one of the reference pulse shapes based on said instantaneous coefficients.

Still further in accordance with the second aspect of the present disclosure, the pulse processing device can for example further comprise calculating a plurality of second distances between the updated average coefficients of the closer one of the reference pulse shapes and the average coefficients of the other ones of the reference pulse shapes, the second distances having a second minimal distance identifying a closer one of said other ones of the reference pulse shapes.

Still further in accordance with the second aspect of the present disclosure, the pulse processing device can for example further comprise, upon determining that said second minimal distance is greater a second distance threshold, registering the instantaneous coefficients as a sparse array of average coefficients of a new reference pulse shape in the reference data.

Still further in accordance with the second aspect of the present disclosure, said generating said sparse array of instantaneous coefficients can for example include from performing one or more discrete transforms on said amplitude values of said pulsed signal.

Still further in accordance with the second aspect of the present disclosure, said performing can for example include performing one or more discrete wavelet transforms on said amplitude values of said pulsed signal, said instantaneous and said average coefficients being wavelet coefficients.

Still further in accordance with the second aspect of the present disclosure, said discrete wavelet transform can for example be a Symmlet-2 discrete wavelet transform.

Still further in accordance with the second aspect of the present disclosure, the pulse processing device can for example have a footprint below 5 cm³, below 2.5 cm³ and most preferably below 1 cm³.

Still further in accordance with the second aspect of the present disclosure, the pulse processing device can for example have a power consumption below 1 mW, preferably below 500 μW and most preferably below 100 μW.

In some embodiments, the classification of a received array of wavelet coefficients into a respective reference pulse shape is performed by comparing geometrical distances between the wavelet coefficients of the received array and average wavelet coefficients associated with some reference pulse shapes. When it is determined that a minimal one of the geometrical distances is below a given distance threshold, the received array of wavelet coefficients can be associated with the corresponding reference pulse shape, which when hardware coded into a standalone integrated circuit or field-programmable gate array can reduce the amount of computational power and/or memory requirements on the pulse processing device compared to existing pulse processing apparatuses.

In accordance with a third aspect of the present disclosure, there is provided a method of sorting coefficients associated to a pulsed signal, the method comprising: sampling a pulsed signal, including generating an array of amplitude values; performing a discrete transform using said amplitude values of said array, including generating an array of coefficients indicative of an energy distribution of said pulsed signal; and sorting said coefficients using a tree sorting algorithm, said tree sorting algorithm finding a maximal one of said coefficients and an N^{th} maximal one of said coefficients, including generating a sparse array of said coefficients consisting of said maximal one of said coefficients, said N^{th} maximal one of said coefficients and any coefficients therebetween, including the relative position of the coefficients relative to one another. For instance, null values may replace each of the other coefficients in said sparse array.

Further in accordance with the third aspect of the present disclosure, N can for example be an integer being smaller than said number of said coefficients of said array.

Still further in accordance with the third aspect of the present disclosure, N can for example be twenty.

Still further in accordance with the third aspect of the present disclosure, said finding can for example comprise disregarding an initial polarity of each of said coefficients, said sparse array comprising the N maximal coefficients with their initial polarity.

Still further in accordance with the third aspect of the present disclosure, a number of said coefficients can for example be equal to or smaller than a number of said amplitude values.

Still further in accordance with the third aspect of the present disclosure, the method can for example further comprise normalizing said N maximal coefficients of said sparse array, the N maximal coefficients ranging between the positive unity and the negative unity.

Still further in accordance with the third aspect of the present disclosure, the method can for example further comprise X-bits quantizing said sparse array of said N maximal coefficients, wherein X is an integer greater than one.

Still further in accordance with the third aspect of the present disclosure, said performing can for example include performing one or more discrete wavelet transforms on said amplitude values of said pulsed signal, said coefficients being wavelet coefficients.

Still further in accordance with the third aspect of the present disclosure, said discrete wavelet transform can for example be a Symmlet-2 discrete wavelet transform.

Still further in accordance with the third aspect of the present disclosure, the Symmlet-2 discrete wavelet transform can for example be a level 4 Symmlet-2 discrete wavelet transform.

In accordance with a fourth aspect of the present disclosure, there is provided a pulse processing device comprising: a substrate; and a pulse compression module mounted on the substrate, the pulse compression module having a processor and a memory having instructions stored thereon that when performed by the processor perform the steps of: sampling a pulsed signal, including generating an array of amplitude values; performing a discrete transform using said amplitude values of said array, including generating an array of coefficients indicative of an energy distribution of said pulsed signal; and sorting said coefficients using a tree sorting algorithm, said tree sorting algorithm finding a maximal one of said coefficients and an N^{th} maximal one of said coefficients, including generating a sparse array of said coefficients consisting of said maximal one of said coefficients, said N^{th} maximal one of said coefficients and any coefficients therebetween, including the relative position of the coefficients relative to one another. For instance, null values may replace each of the other coefficients in said sparse array.

Further in accordance with the fourth aspect of the present disclosure, N can for example be an integer being smaller than said number of said coefficients of said array.

Still further in accordance with the fourth aspect of the present disclosure, N can for example be twenty.

Still further in accordance with the fourth aspect of the present disclosure, a number of said coefficients can for example be equal to or smaller than a number of said amplitude values.

Still further in accordance with the fourth aspect of the present disclosure, said finding can for example further comprise disregarding an initial polarity of each of said coefficients, said sparse array comprising the N maximal coefficients with their initial polarity.

Still further in accordance with the fourth aspect of the present disclosure, the pulse processing device can for example further comprise normalizing said N maximal coefficients of said sparse array, the N maximal coefficients ranging between the positive unity and the negative unity.

Still further in accordance with the fourth aspect of the present disclosure, the pulse processing device can for example further comprise X-bits quantizing said sparse array of said N maximal coefficients, wherein X is an integer greater than one.

Still further in accordance with the fourth aspect of the present disclosure, said performing can for example include performing one or more discrete wavelet transforms on said amplitude values of said pulsed signal, said coefficients being wavelet coefficients.

Still further in accordance with the fourth aspect of the present disclosure, said discrete wavelet transform can for example be a Symmlet-2 discrete wavelet transform.

Still further in accordance with the fourth aspect of the present disclosure, said Symmlet-2 discrete wavelet transform can for example be a level 4 Symmlet-2 discrete wavelet transform.

Still further in accordance with the fourth aspect of the present disclosure, the pulse processing device can for example have a footprint below 5 cm³, below 2.5 cm³ and most preferably below 1 cm³.

Still further in accordance with the fourth aspect of the present disclosure, the pulse processing device can for example have a power consumption below 1 mW, preferably below 500 μW and most preferably below 100 μW.

In some embodiments, the above-described electrical pulses may be generated by an entity (e.g., brain tissue, heart tissue) which has been previously stimulated with a stimulation signal. In these embodiments, the pulse processing device can decide how the pulse generating entity is to be stimulated. For instance, the stimulation signal may be a given optical signal of a given colour and/or a given intensity, as in optogenetic experiments, or an electrical signal. The stimulation of the pulse generating entity can trigger the generation of one or more electrical signals that are to be detected, compressed and classified on the go by the pulse processing entity, and so forth. When all these steps are performed using a subject-mounted standalone pulse processing device, the pulses processing device operates in a closed-loop, which can be beneficial in at least some situations. For example, the pulse generating entity may be stimulated with a given stimulation signal which is expected to lead to a corresponding reaction from the pulse generating entity. The corresponding reaction may be recognized by the generation of electrical pulses of a given reference pulse shape following the stimulation. When it is determined that at least some pulses are detected within a given time window following the stimulation, and classified to be associated with that given reference pulse shape, the stimulation may be deemed to be successful. As a result, the pulse generating entity may be controlled in closed-loop using the pulse processing device.

In accordance with a fifth aspect of the present disclosure, there is provided a method of stimulating a pulse generating entity, the method comprising: implanting a probe head within the pulse generating entity, the probe head having a stimulation element and an electrode; during a given time window, the electrode collecting a corresponding plurality of electrical signals; and upon associating at least a threshold number of said electrical signals to a common reference pulse shape, stimulating the pulse generating entity using the stimulation element, including generating a stimulation signal within the pulse generating entity.

Further in accordance with a fifth aspect of the present disclosure, said stimulation signal can for example be selected on the basis of the common reference pulse shape of the threshold number of electrical signals.

Still further in accordance with a fifth aspect of the present disclosure, said stimulation signal can for example be an optical stimulation signal.

Still further in accordance with a fifth aspect of the present disclosure, a wavelength of said optical stimulation signal can for example depend on the common reference pulse shape of the threshold number of electrical signals.

Still further in accordance with a fifth aspect of the present disclosure, said given time window can for example extend during at least 100 ms, preferably at least 50 ms and most preferably at least 25 ms.

Still further in accordance with a fifth aspect of the present disclosure, said stimulation signal can for example be associated with a reaction of said pulse generating entity, the method further comprising conditioning the pulse generating entity to react according to the reaction via said stimulating.

Still further in accordance with a fifth aspect of the present disclosure, said pulse generating entity can for example be at least one of brain tissue and heart tissue.

Still further in accordance with a fifth aspect of the present disclosure, the method can for example further comprise accessing reference data having a plurality of reference pulse shapes, the common reference pulse shape being identifiable in said reference data.

In accordance with a sixth aspect of the present disclosure, there is provided a pulse processing device comprising: a substrate; a probe head implantable within a pulse generating entity, the probe head having a stimulation element and an electrode; a stimulation module mounted on the substrate and communicatively coupled to the probe head, the stimulation module having a processor and a memory having instructions stored thereon that when performed by the processor perform the steps of: during a given time window, collecting a plurality of electrical signals using said electrode; and upon associating at least a threshold number of said electrical signals to a common reference pulse shape, stimulating the pulse generating entity using the stimulation element, including generating a stimulation signal within the pulse generating entity.

Further in accordance with the sixth aspect of the present disclosure, said stimulation signal can for example be selected on the basis of the common reference pulse shape of the threshold number of electrical signals.

Still further in accordance with the sixth aspect of the present disclosure, said stimulation element can for example include an optical fiber delivering an optical stimulation signal to the pulse generating entity.

Still further in accordance with the sixth aspect of the present disclosure, a wavelength of said optical stimulation signal can for example depend on the common reference pulse shape of the threshold number of electrical signals.

Still further in accordance with the sixth aspect of the present disclosure, said given time window can for example extend during at least 100 ms, preferably at least 50 ms and most preferably at least 25 ms.

Still further in accordance with the sixth aspect of the present disclosure, said stimulation signal can for example be associated with a reaction of said pulse generating entity, the stimulating module further performing conditioning the pulse generating entity to react according to the reaction via said stimulating.

Still further in accordance with the sixth aspect of the present disclosure, the pulse processing device can for example further comprise accessing reference data having a plurality of reference pulse shapes, the common reference pulse shape being identifiable in said reference data.

Many further features and combinations thereof concerning the present improvements will appear to those skilled in the art following a reading of the instant disclosure.

### DESCRIPTION OF THE FIGURES

In the figures,

FIG. 1 is a schematic view of an example of a pulse processing device, in accordance with one or more embodiments;

FIG. 2 is an oblique view of the pulse processing device of FIG. 1, shown with a probe implanted into brain tissue, and a controller, in accordance with one or more embodiments;

FIG. 2A is an exploded view of the probe and the brain tissue of FIG. 2, in accordance with one or more embodiments;

FIG. 2B is a schematic view of the probe of FIG. 2A, shown with an example stimulation element emitting a stimulation signal, and example detection electrodes detecting neural pulses on the go, in accordance with one or more embodiments;

FIG. 3 is a schematic view of an example of a computing device of the controller of FIG. 2, in accordance with one or more embodiments;

FIG. 4 is a block diagram of an example of a pulse processing device, showing a detection module, a compression module and a classification module, in accordance with one or more embodiments;

FIG. 5 is block diagram of an example control sequence used for interfacing the modules of FIG. 4, in accordance with one or more embodiments;

FIG. 6 is a graph showing detected amplitude values as a function of time, showing a plurality of raw neural pulses compared to a threshold, in accordance with one or more embodiments;

FIG. 7 is a block diagram of the detection module of FIG. 4, in accordance with one or more embodiments;

FIG. 8A is a schematic view of an example of an array of amplitudes values, and a plurality of arrays of wavelet coefficients obtained by performing a series of discrete wavelet transforms on the array of amplitude values, in accordance with one or more embodiments;

FIG. 8B is a block diagram showing a discrete wavelet transform submodule, a wavelet coefficient selection submodule and a wavelet coefficient compression submodule, the submodules being part of the compression module of FIG. 4, in accordance with one or more embodiments;

FIG. 8C is graph of a plurality of detected arrays of amplitude values and associated reference pulse shapes as determined by the compression module of FIG. 4, in accordance with one or more embodiments;

FIG. 9 is a flow chart of a method of compressing an array of amplitudes values into an array of wavelet coefficients, in accordance with one or more embodiments;

FIG. 10 is a block diagram of an example compression module of the pulse processing module of FIG. 4, in accordance with one or more embodiments;

FIG. 11 is a flow chart of a method of associating an array of wavelet coefficients to a corresponding reference pulse shape, in accordance with one or more embodiments;

FIG. 12 is a block diagram of an example classification module of the pulse processing module of FIG. 4, in accordance with one or more embodiments;

FIG. 13 is a flow chart of a method of controlling a pulse generating entity via a stimulation signal, in accordance with one or more embodiments;

FIG. 14 is a block diagram of an example decision making module of the pulse processing module of FIG. 4, in accordance with one or more embodiments;

FIG. 15 are images of an example of an hardcoded integrated circuit of the pulse detection module, the pulse compression module, the classification module, the decision making module, and the stimulation module of the pulse processing device of FIG. 4, in accordance with one or more embodiments;

FIG. 16A is a graph showing receiver operating characteristic (ROC) curves for a 6-dB signal with stimulated with a varying firing rate, in accordance with one or more embodiments;

FIG. 16B is a graph showing a subset of the neural signal used to compute the ROC curves of FIG. 16A, in accordance with one or more embodiments;

FIG. 16C is a graph showing cost-function (CF) scores for the pulse processing device of FIG. 4 compared to existing pulse processing apparatuses, in accordance with one or more embodiments;

FIG. 17A is a graph showing power consumption of the pulse processing device of FIG. 4 for stimulation with five different firing rates, in accordance with one or more embodiments;

FIG. 17B is a graph showing the current consumption of a wireless transceiver of the pulse processing device of FIG. 4 with and without compression at an on-air data rate of 2 Mbps, in accordance one or more embodiments;

FIG. 17C is a graph showing the current consumption of a wireless transceiver of the pulse processing device using the pulse processing device with and without compression at an on-air data rate of 250 Mbps, in accordance one or more embodiments;

FIG. 18A is a graph showing classification results using the method of FIG. 11 versus existing techniques, in accordance with one or more embodiments;

FIG. 18B is a graph showing unsupervised classification results of the method of FIG. 11 compared with K-means, in accordance with one or more embodiments;

FIG. 18C is a graph showing normalized classification performance according to a threshold T₁, in accordance with one or more embodiments;

FIG. 18D is a graph showing classification results as a function of the signal's signal to noise ratio (SNR), in accordance with one or more embodiments;

FIG. 19A is a pie chart breaking down the power consumption for each modules of the pulse processing device of FIG. 4, in accordance with one or more embodiments;

FIG. 19B is a graph showing power consumption as a function of different neural firing rates and power consumption as a function of the operating frequency of the modules of the pulse processing device of FIG. 4, in accordance with one or more embodiments;

FIG. 20A is a graph of an example of a reconstructed signal using the detected, compressed and sorted wavelet coefficients processed with the pulse processing device of FIG. 4 as it is mounted to a rat's brain tissue, in accordance with one or more embodiments;

FIGS. 20B and 20c are graphs showing detected, compressed and sorted arrays of wavelet coefficients at two different depths within the rat's brain tissue, in accordance with one or more embodiments;

FIG. 21A is a graph of an example of a reconstructed neural signal and closed loop (CL) stimulation triggers acquired with a freely-moving mouse expressing a ChR2-mCherry in inhibitory neurons of the prelimbic cortex, in accordance with one or more embodiments;

FIG. 21B is a graph showing two signal close-ups showing the CL stimulation pulses and evoked silencing phase due to the ChR2-mCherry activation, in accordance with one or more embodiments;

FIG. 21C is a graph showing two CL window enlargements with and without CL stimulation and trigger, in accordance with one or more embodiments;

FIG. 21D are graphs showing AP sorted in real-time by the pulse processing device of FIG. 4 compared to being sorted by a PCA with K-means, in accordance with one or more embodiments;

FIG. 22A is a graph showing electrical pulses as a function of time, with optical stimulation signals and following silencing durations taken during a control experiment, in accordance with one or more embodiments;

FIG. 22B is a graph showing CL stimulation performed in a non-injected mouse, with no silencing durations, in accordance with one or more embodiments;

FIG. 22C are confocal images of the mouse used in the in vivo validation (11 months post infection) showing the ChR2-mCherry expression (red) and the nuclei labeled with DAPI (blue), in accordance with one or more embodiments;

FIG. 23 is a graph showing in situ classification performed by the pulse processing device of FIG. 4 versus offline classification involving PCA with K-means for a number of freely-moving mouse experiments, in accordance with one or more embodiments;

FIG. 24A is a graph showing the evolution of an in situ computed threshold versus an offline windowed MAD, superposed over a raw neural signal, in accordance with one or more embodiments;

FIG. 24B is a graph showing the threshold standard deviation from its mean vale for a number of freely-moving mouse experiments, in accordance with one or more embodiments;

FIG. 25A is a graph showing the mean signal-to-noise distortion (SNDR) for a number of different experiments, in accordance with one or more embodiments; and

FIG. 25B are graphs showing action potential (AP) waveforms superimposed on their original raw electrical signal, in accordance with one or more embodiments.

### DETAILED DESCRIPTION

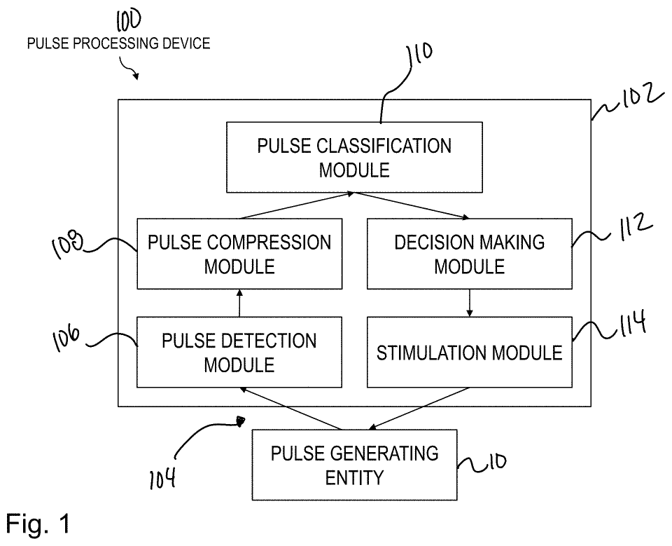

FIG. 1 shows an example of a pulse processing device 100 which can stimulate a pulse generating entity 10 and detect pulses generated thereby. In some embodiments, the pulse generating entity 10 includes biological tissue. For instance, the pulse generating entity 10 may include brain tissue in some embodiments whereas the pulse generating entity 10 may include heart tissue in some other embodiments.

As depicted, the pulse processing device 100 has a controller 102 which is communicatively coupled to a probe 104 which is to be implanted into the pulse generating entity 10.

In this example, the controller 102 has a pulse detection module 106, a pulse compression module 108, a pulse classification module 110, a decision making module 112 and a stimulation module 114. The decision making module 112 may be configured to determine how, when and at which frequency the pulse generating entity 10 is to be stimulated. As shown, the stimulation module 114 is communicatively coupled to the decision making module 112. Accordingly, based on the decisions taken by the decision making module 112, the stimulation module 114 may be configured to generate instructions to stimulate the pulse generating entity 10 with a given stimulation signal via the probe 104. For instance, the stimulation signal may be an optical signal of a given amplitude, duration, and/or color (wavelength). The stimulation signal may be an electrical stimulation signal of a given amplitude, pulse shape, duration and/or frequency.

In some embodiments, the pulse generating entity 10 may generate electrical pulses of different pulse shapes in short succession. When successive electrical pulses are detected within a given time window using the pulse detection module, and classified to be of the same reference pulse shape, the stimulation module 114 may stimulate the pulse generating entity 10 with a corresponding stimulation signal to condition the pulse generating entity 10.

To save computational power and/or memory requirements, the detected pulses are processed to compress them and to classify them into a corresponding reference pulse shape. Based on the reference pulse shape of the detected pulsed signal, the decision making module 112 may determine whether an experiment is successful or with which stimulation signal the pulse generating entity 10 is to be stimulated. In any case, the modules 106 through 114 form a closed loop which may be repeated any desired number of times, depending on the embodiment.

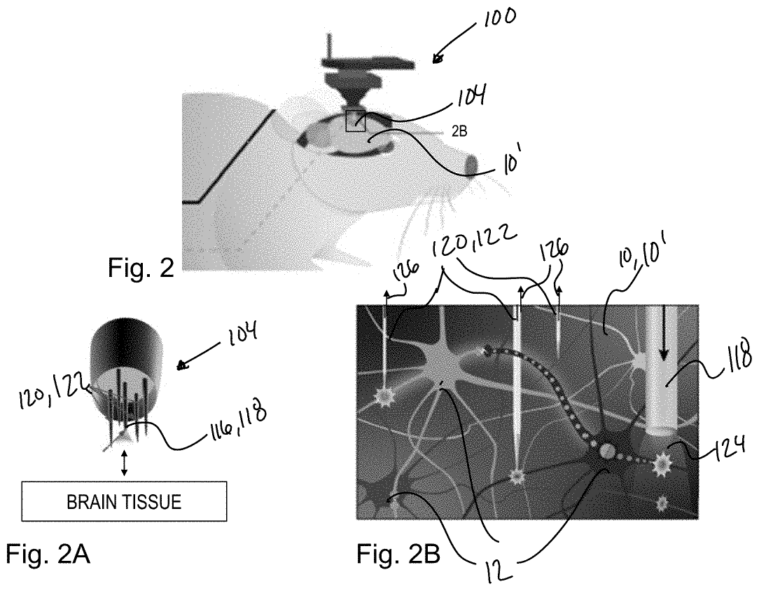

FIG. 2 shows an example of the pulse processing device 100, with the probe 104 being implanted into a rat's brain tissue 10′. As best shown in FIG. 2A, the probe 104 has one or more stimulation elements 116 emitting one or more stimulation signals. As shown in this specific embodiment, the stimulation element 116 is an optical fiber 118 carrying an optical stimulation signal. For instance, the optical signal may be at a given wavelength and carry a given amount of optical energy. In some other embodiments, the stimulation element 116 may be a conductive tip emitting an electrical stimulation signal. The probe 104 also has one or more electrodes 120 collecting electrical signals generated by the mouse/rat's brain tissue 10′. As shown, the electrodes 120 are provided in the form of conductive tips 122 which may be positioned to penetrate at different depths within the brain tissue 10′ relative to the stimulation element 118. The probe 104 can also have one or more optical fibers to collect any optical signals that could be generated by the mouse/rat's brain tissue 10′. As such, the stimulation signal may be electrical, optical, chemical and the like. FIG. 2B shows an example where the optical fiber 118 is used to emit an optical stimulation signal 124. The optical stimulation signal is perceived by surrounding neuron(s) 12 which may generate electrical signal(s) 126 as a response. In this specific embodiment, the electrodes 120, which are distributed about the optical fiber 118, collect the generated electrical signals 126 for further processing by the controller 102.

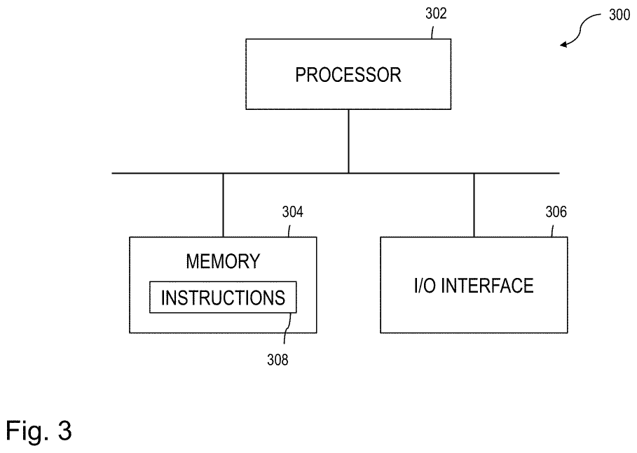

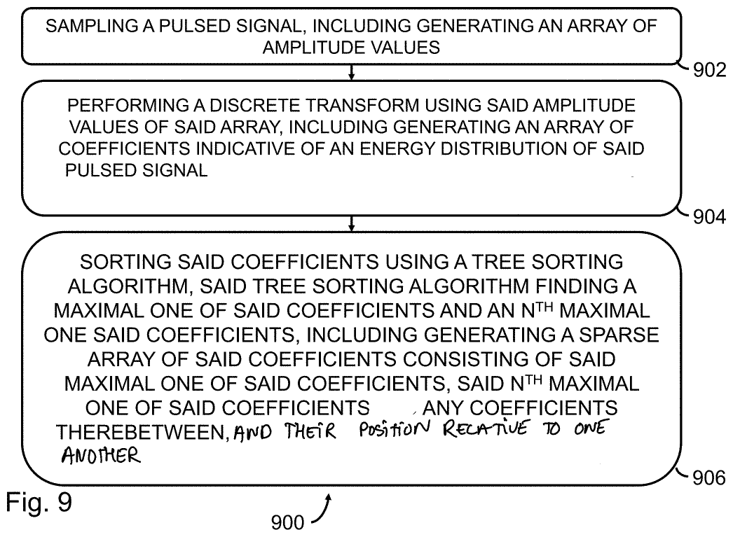

The controller 102 can be provided as a combination of hardware and software components. The hardware components can be implemented in the form of a computing device 300, an example of which is described with reference to FIG. 3. Moreover, the software components of the controller 102 can be implemented in the form of a software circuits, flow charts of which are described with reference to FIGS. 9, 11 and 13.

Referring to FIG. 3, the computing device 300 can have a processor 302, a memory 304, and I/O interface 306. Instructions 308 for processing the detected signals and/or stimulating the pulse generating entity can be stored on the memory 304 and accessible by the processor 302.

The processor 302 can be, for example, a general-purpose microprocessor or microcontroller, a digital signal processing (DSP) processor, an integrated circuit, a field programmable gate array (FPGA), a reconfigurable processor, a programmable read-only memory (PROM), or any combination thereof.

The memory 304 can include a suitable combination of any type of computer-readable memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 306 enables the computing device 300 to interconnect with one or more input devices, such as button(s), switch(es), or with one or more output devices such as a communication unit (e.g., an antenna).

Each I/O interface 306 enables the controller 102 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. WMAX), SS7 signaling network, fixed line, local area network, wide area network, and others, including any combination of these.

The computing device 300 and the software circuits described herein are meant to examples only. Other suitable embodiments of the controller 102 can also be provided, as it will be apparent to the skilled reader.

With the rise of optogenetics in experimental neuroscience methods, it is now possible to use light to study the microcircuits deep inside the intact brain. This groundbreaking approach can activate specific neurons in the cortex of transgenic animals to observe their role in vast biological networks. Recently, optogenetics has been used to carry various behavioural experiments with freely behaving transgenic rodents, especially with mice. These experiments are performed using predefined stimulation patterns, with or without, simultaneous electrophysiological recording, and often using a bulky hardwired setup.

A promising paradigm with potential to accelerate the development of new therapeutics against brain diseases consists of closing the loop between the optical stimulator and the neural sensing device. This approach, known as closed-loop optogenetics, can stimulate select brain areas according to the level of activity detected from a specific group of neurons or brain areas. Mainly, it requires three essential components, including a neural recording interface, an optical stimulator, and a neural analyzer. One major challenge facing CL optogenetic systems consists of analyzing dense neural activity in real time to quickly issue proper stimulation feedback commands inside specific brain areas. Existing pulse processing apparatuses designed for this purpose typically use a PC-based system and a wired experimental setting, which usually limits their use to anesthetized animals models. Although these systems are convenient for performing complex neural analysis, they are not suitable for freely-moving experiments seeking natural behaviour, since the animals are tethered.

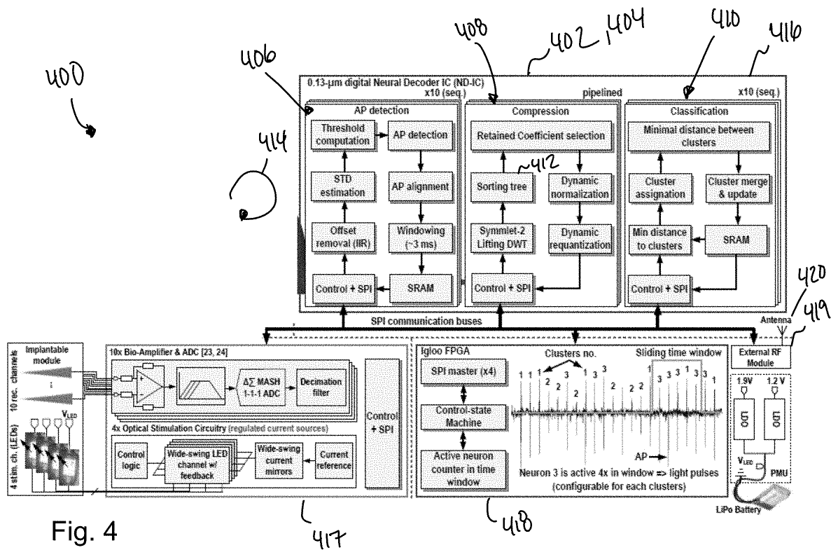

FIG. 4 shows another example of a pulse processing device 400 that can be used in such optogenetics applications, in accordance with a specific embodiment. The pulse processing device 400 is provided in the form of a wireless electro-optic headstage which uses a 0.13-m CMOS custom integrated circuit (IC) 402 implementing a digital neural decoder (ND-IC) 404 for enabling real-time closed-loop (CL) optogenetics. In this example, the headstage is sufficiently light and small to be mountable to a rat's head without preventing the rat from normal behaviour. The headstage can incorporate a battery or battery pack which can power the pulse processing device 400 for a suitable amount of time.

The ND-IC 404 processes the neural activity data using three digital modules: 1) a detector module 406 detecting and extracting the action potential (AP) of individual neurons using an adaptive threshold; 2) a data dompression module 408 compressing the detected AP using a discrete transform such as the Symmlet-2 discrete wavelet transform (DWT) for reducing the amount of data to be communicated within the ND-IC 404 and also communicated to external network using a low-power wireless link, for instance; 3) a classification module 410 sorting the compressed AP into separated reference shapes on the fly according to their pulse shapes.

As will be described below, the compression module 408 reduces the complexity from O(n²), to O(n·log (n)) compared to existing pulse processing apparatuses, while using two times less memory, thanks to the use of a new coefficient sorting tree 412. Accordingly, the compression module 408 discussed herein can have reduced electrical power requirements and/or reduced computational power requirements. The AP classification module 410 can reuse both the compressed DWT coefficients to perform implicit dimensionality reduction, which allows for performing intensive signal processing on-chip, while increasing power and hardware efficiency. The classification module 410 can also reuse the signal standard deviation already computed by the AP detector module 406 as threshold for performing automatic AP sorting. The headstage also introduces a wireless CL module 414 between the neural data acquisition module and the optical stimulator. The proposed CL module 414 uses the AP sorting and timing information produced by the ND-IC 404 for detecting complex firing patterns within the brain. The headstage is also smaller (1.13 cm³), lighter (3.0 g with a 40 mAh battery) and less-invasive than existing pulse processing apparatuses, while providing a measured autonomy of 2h40, with the ND-IC 404. Moreover, the ND-IC 404 is provided within a housing 416 which is miniature, lightweight and wireless, which is well suited for CL optogenetic experiments in real time. Further, the pulse processing device 400 has been validated in vivo and it is demonstrated that the CL scheme with a freely-moving animal works in some circumstances. More specifically, the whole device 400 and the ND-IC 404 are firstly validated in vivo in the LD thalamus of a Long-Evans rat, and then, in freely-moving CL experiments involving a mouse virally expressing ChR2-mCherry in inhibitory neurons of the prelimbic cortex, and the results show that the proposed pulse processing device 400 can work well within an in vivo experimental setting with a freely moving mouse.

The ND-IC 404 is the most important module for CL optogenetics in the pulse processing system 400. It provides real-time neural data decoding to establish a connection between a readout and a stimulator. As shown in the block diagram of FIG. 4, the IC 404 has the three main modules: the detection module 406, the compression module 408 and the classification module 410. As discussed briefly above, the detector module 406 detects and extracts the AP of individual neurons. It uses an adaptive threshold based on real-time signal analysis performed through a feedback loop with a simplified implementation. The compression module 408 is using a Symmlet-2 (Symmlet-2) discrete wavelet transform (DWT) lifting scheme followed by dynamic coefficient discrimination using a sorting tree and dynamic coefficient re-quantization scheme for neural data compression. Each AP waveform is compressed for saving power and bandwidth, and transmitted wirelessly along with the classification ID for live monitoring. Additionally, the data compression allows to operate the wireless transceiver in low-data rate mode, which extends the lifespan and increases the transmitting range. The classification module 410 sorts the AP on-the-fly according to their wave shapes. For performing automatic classification, the classification module 410 can reuse i) a subset of the DWT coefficients from the compression module, and ii) the estimated signal's standard deviation calculated by the detector module 406.

In addition to the ND-IC 404, the headstage encompasses three other building blocks: i) a custom mixed-signal 0.13-m CMOS system-on-chip (SoC) 417 for simultaneous neural recording and optogenetic stimulation, which is depicted in FIG. 4. It includes all the circuits required to perform multichannel neural recording and optogenetic stimulation. It allows for conditioning and sampling the low-amplitude extracellular AP over 10 channels in parallel and to perform optical stimulation with a 4-ch LED driver circuit performing precise current regulation, ii) a ultra low-power Igloo AGLN250 FPGA (5×5 mm² BGA) controller 418 from Microsemi, USA, and iii) a low-power nRF24L01p wireless transceiver 419 from Nordic Semiconductor, Norway. The wireless transceiver 419 is connected to a 2.4 GHz ceramic antenna 420, and is configured to operate at 0 dBm and at either 250 kbps, 1 Mbps or 2 Mbps (user selectable), which can be set according to the required power consumption and range of the system. A 1.9-V low-dropout regulator (LDO) provides the voltage for the wireless transceiver and for one of the FPGA IO banks, while another 1.2-V LDO provides the voltage for the rest of the system.

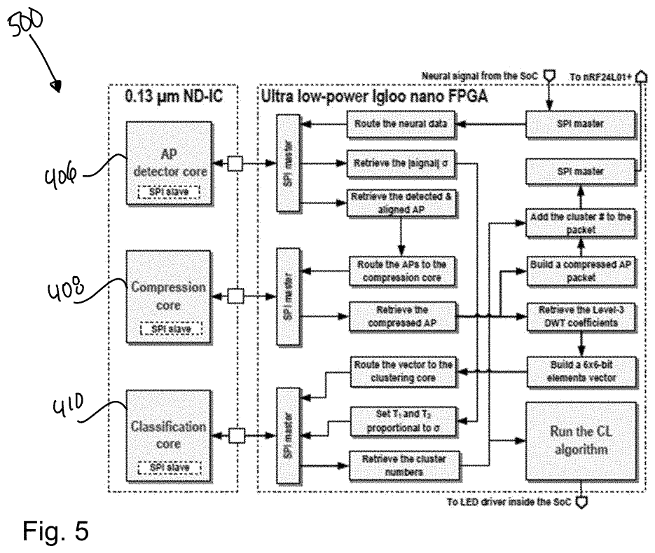

Still referring to FIG. 4, the FPGA controller 418 is used for interfacing with the proposed ND-IC chip 404, the mixed-signal SoC 417 and the wireless transceiver 419. The use of the FPGA controller 418 provides all the necessary flexibility to ease system integration. An Igloo FPGA from Microsemi was selected for its low power consumption and its small footprint. This controller is interfaced with the ND-IC chip and with the other building blocks using dedicated master SPI modules. The FPGA control sequence 500 is depicted in FIG. 5. Mainly, the neural data is routed from the mixed-signal SoC readout to the ND-IC detector module, then the detected APs are forwarded to the ND-IC compression module, and finally a subset of the compressed waveforms DWT coefficients are routed to the ND-IC classification module. After performing data decoding, the sorted neural data (cluster number, timestamp and compressed samples) are encapsulated into data packets and sent to the wireless transceiver. Meanwhile, the cluster number and AP timestamp are routed to the CL algorithm. When a CL stimulation is required, the FPGA controls the light pulse sequence by communicating with the mixed-signal SoC.

The detection module, the compression module, the classification module and the stimulation module are described in this order in the following paragraphs.

The proposed detector module 406 has two main modules: the pre-processor for conditioning the neural data and the adaptive threshold for discriminating the APs from noise.

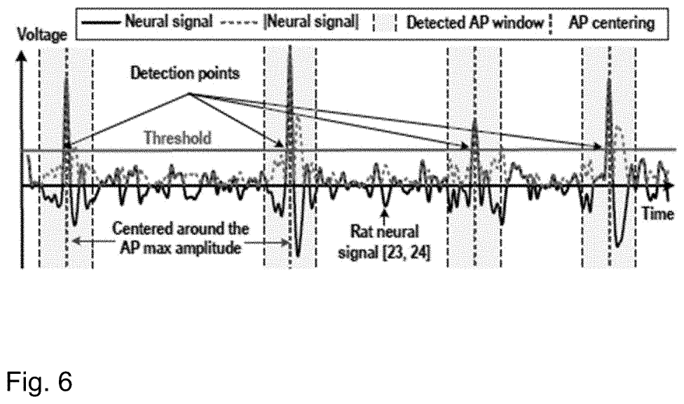

The detector module 406 is based on the absolute value operator. As previously demonstrated, this simplified operator can be as efficient as a more sophisticated energy-based operator, such as the non-linear energy operator (NEO), the smoothed NEO and the matched-filter (MF), providing an excellent trade-off between low complexity and high performance. The absolute value operator concept is shown in FIG. 6, showing the neural signal in solid line whereas the absolute value of the neural signal is shown in the dashed line. Upon threshold crossing, the APs are extracted and aligned on their peak amplitude sample.

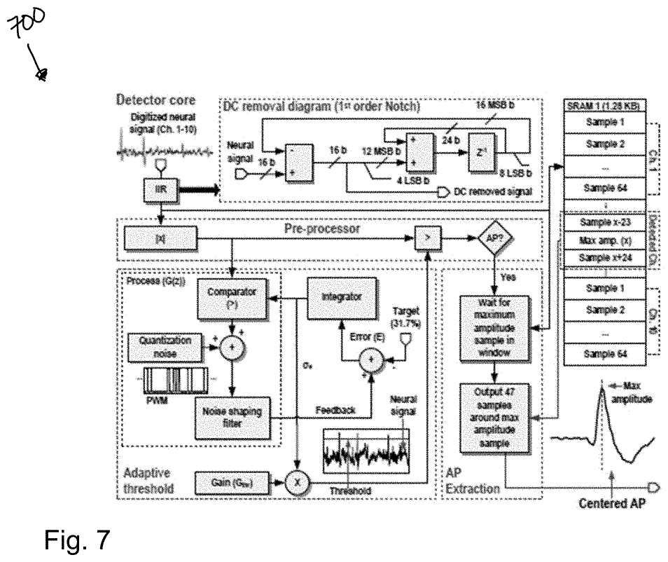

FIG. 7 shows a block diagram of an example detector module 700. First, the neural signal is high-pass filtered by an infinite impulse response (IIR) filter to remove any DC level. A power and area efficient 1^{st} order notch filter is implemented. This filter uses bit pruning and addition/subtraction operations only. Upon signal threshold crossing, the peak sample is found across a 31-sample window immediately starting after the detection point. Then, a frame of 48 samples including the peak at the middle is forwarded to the compression module. Such an alignment around the peak value is used to facilitate AP classification, given than two APs emitted from the same neuron might appear to belong to different clusters if they were misaligned and not properly extracted.

The output of the absolute value operator is compared with a threshold, the value of which is recalculated in real time according to the characteristics of the neural signal. The proposed threshold calculation strategy is based on the estimated standard deviation (σₐ) of the noise embedded in the neural signal. This strategy is effective and robust, and is used extensively in electrophysiology. The detector module computes σₐ through a feedback loop that is using the statistical property of the signal's noise, as seen in FIG. 7. The adaptive threshold computation technique is adapted for an efficient CMOS implementation within the ND-IC, and can change from one embodiment to another.

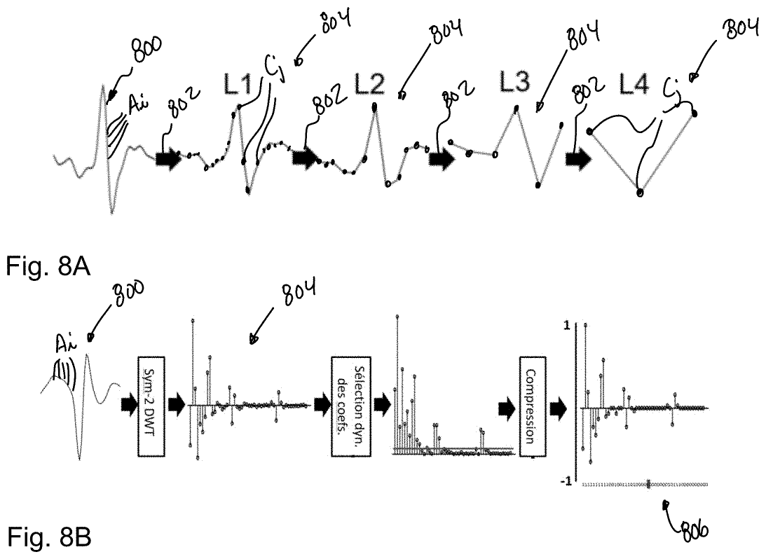

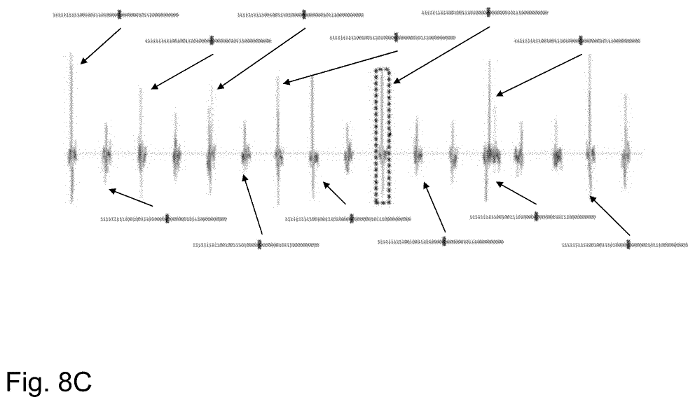

As mentioned above, the detected AP is provided in the form of an array 800 of amplitude values Ai, such as the one shown on the leftmost waveform of FIG. 8A, with i varying from 1 to N, with N denoting an integer representative of the number of data points in the array 800. In this specific embodiment, N may be 48. As depicted, to reduce the computational power and memory requirements imparted on the pulse processing device, any array of amplitude values generated by the detection module may be compressed by performing one or more discrete transforms 802. For instance, the discrete transforms can be embodied in a hardware circuit by a series of high-pass filters, low-pass filters and the like. As shown, one or more discrete transforms 802 can be performed based on the array 800 of amplitude values Ai, which in turn can generate corresponding arrays 804 of coefficients Cj which are indicative of an energy distribution of the detected AP. As shown, the numbers of coefficients Cj of the arrays 804 are smaller than a number of the amplitude values Ai of the original array 800, thereby actually compressing the data that need to be computed and/or stored on the go. In the present disclosure, the discrete transform is generally provided in the form of a discrete wavelet, with the coefficients Cj being in fact wavelet coefficients.

As best shown in FIG. 8B, the received array 800 of amplitude values Ai is transformed via one or more discrete wavelet transforms to provide a first array 804 of wavelet coefficients. Then, some of the wavelet coefficients are selected based on the amount of information pertaining to energy distribution and/or frequency that that each wavelet coefficient carries. After the selection process has been performed, the remaining wavelet coefficients are sparsed in a manner that wavelet coefficients of significant value are kept whereas wavelet coefficients of lesser importance are zeroed, to finally provide a string 806 of bits which at least partially represent the original detected array 800 of amplitude values Ai. As such, as a series of AP are being detected by the detection module, the compression module may compress the detected AP on the go thereby transforming a plurality of arrays of amplitude values into a corresponding plurality of smaller, more easily processable arrays of wavelet coefficients, such as shown in FIG. 8C. It is understood that the zeroes of the strings shown in FIG. 8C are used to encode the position of each of the selected coefficients relative to one another.

FIG. 9 shows a method 900 of sorting coefficients associated to a pulsed signal. As shown, the method 900 has a plurality of method steps which can be hardcoded in a printed circuit board in some embodiments.

At step 902, a pulsed signal is sampled. The sampling of the pulsed signal leads to the generation of an array of amplitude values Ai. The pulsed signal may be sampled by the detection module discussed above, after which the detected pulsed signal is transmitted to the compression module.

At step 904, a discrete transform is performed using the amplitude values Ai of the array sampled at step 902. This step includes the generation of an array of coefficients Ci indicative of an energy distribution of the pulsed signal sampled at step 902. As with such discrete transforms, the number of the coefficients Ci can be the same as a number of the amplitude values Ai. In some other embodiments, the number of the coefficients Ci can be smaller than a number of the amplitude values Ai. Reducing the dimension from the array of amplitudes values Ai to the array of coefficients Ci can reduce the power and/or memory requirements of the pulse processing device.

In some embodiments, the discrete transforms are provided in the form of discrete wavelet transforms. In such embodiments, the coefficients resulting from the discrete wavelet transforms are wavelet coefficients. As different types of discrete wavelet transform may be used, the Symmlet-2 discrete wavelet transform was found to be convenient in at least some circumstances.

At step 906, the coefficients Ci are sorted using a tree sorting algorithm. In this embodiment, the tree sorting algorithm finds a maximal one of the coefficients Ci, Cmax and an N^{th} maximal one of the coefficients Ci, Cn. Wth these coefficients found, step 906 also includes the generation of a sparse array of coefficients Ci which consists of the maximal coefficient Cmax, the N^{th} maximal coefficient Cn and any coefficients therebetween, with null values replacing each of the other coefficients in the sparse array. As such, the N coefficients Ci which carry the more relevant information pertaining to the corresponding AP are kept along with their position relative to one another. The information of their relative positions can be provided in the form of zeroes replacing each one of the other, non-kept coefficients. In this way, the burden in terms of computational and/or memory requirements can be reduced.

It is expected that the number N of coefficients that are selected at step 906 is generally smaller than the number of coefficients in the array. Accordingly, at least some coefficients are zeroed to provide the sparse array of coefficients Cmax, Cmax-1, . . . , Cn. In some embodiments, the number N of coefficients is set to at least 30, preferably set to at least 25 and most preferably set to at least 20. In some embodiments, the detected array of amplitudes values comprises 48 data points, which when compressed using the method 900 result in only 6 relevant data points, leaving zeroes on the 42 addresses of the array.

It is noted that, at least in some embodiments, the step 906 includes a step of calculating an absolute value of all the coefficients Ciat lea, and the step of finding the coefficients Cmax, Cmax-1, . . . , Cn is based on the absolutes values. As such, the initial polarity (e.g., whether the coefficients are positive or negative) may be disregarded in the selection step. However, as the polarity of the coefficients Cmax, Cmax-1, . . . , Cn may be disregarded in the selection step, the resulting sparse array of coefficients will include the initial polarity of each of the N maximal coefficients so as to properly convey the energy distribution of the detected AP. In some embodiments, the method 900 may have a step of normalizing the N maximal coefficients of the array between the position unity and the negative unity, i.e., between −1 and +1. The normalization can be performed by dividing each one of the Cmax, Cmax-1, . . . , Cn coefficients by the maximal coefficient Cmax. In some embodiments, the method 900 may have a step of quantizing the coefficients of the array into a string of a given number of bits.

It is envisaged that the controller incorporating the pulse compression module, or the pulse compression module itself, can have a footprint below 5 cm³, below 2.5 cm³ and most preferably below 1 cm³, with a power consumption below 1 mW, preferably below 500 μW and most preferably below 100 μW.

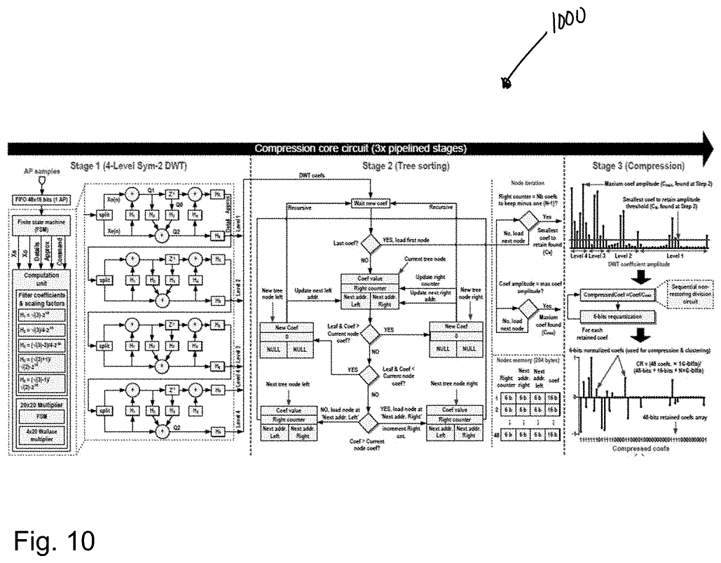

FIG. 10 shows a detailed example of such a compression module 1000. As depicted, the AP compression module 1000 has three pipelined stages. The first stage includes a 4-Level Symmlet-2 lifting sequential DWT which is performed on each detected AP in this specific embodiment. However, any other suitable discrete transform could have been used instead of the 4-Level Symmlet-2 DWT. The second stage processes the coefficients outputted by the first stage according to the sorting tree described with reference to FIG. 9, in order to find the Cmax, Cmax-1, . . . , Cn coefficients. The third stage performs a compression by dynamically thresholding the DWT coefficients to keep only N of them. Further compression is provided using a dynamic re-quantization operation in this specific embodiment.

The detected APs are forwarded to the compression module, which can increase the number of recording channels, reduce the power consumption, and increase the transceiver range, while providing a dimensionality reduction to help the classification module. As shown in FIG. 10, the DWT-based compression technique uses three pipelined stages. First, a 4-Level Symmlet-2 lifting DWT is applied, followed by a coefficient sorting stage, and finally by a compression stage. The proposed CMOS implementation in this example requires much less circuit area, memory (2×), and power (˜75×), than when implemented in an FPGA.

More specifically, in the first stage, a Symmlet-2 lifting DWT is performed on each of the detected AP waveforms. The Symmlet-2 lifting scheme diagram is shown on the left-hand side of FIG. 10, and is performed sequentially using a first in first out (FIFO), a finite stage machine (FSM) and a computation unit. When a new AP becomes available inside the FIFO, the FSM begins the lifting sequence by sending commands—along with the AP samples—to the computation unit. Within this unit, multiplications involving the filter coefficients are performed with a simplified custom 16×20 bits multiplier circuit, consisting of an FSM and a 4×20 Wallace tree. This topology can save significant circuit area (˜80% smaller compared to some existing pulse processing apparatuses). The filter coefficients and scaling factors H₁-H₆, (values provided in FIG. 10), are stored inside general-purpose constant registers within the computation unit. The lifting FSM routes the coefficients to the next DWT level as well as each detailed coefficient to the next coefficient sorting stage, so the coefficients can be sorted while the lifting is being performed.

The proposed compression module 1000 uses an advantageous sorting tree circuit to find the coefficient having the N^{th} amplitude (C_{N}). As shown in the middle portion of FIG. 10, the sorting tree circuit has 48 nodes, each of which encompasses different memory space, including a DWT coefficient, the number of greater coefficients, and two memory pointers. The left and right memory pointers each link to the next node with a smaller or greater coefficient amplitude, compared to the current node coefficient. The tree is gradually filled, as the coefficients are produced by the lifting circuit, and the new nodes are stored within a 204 bytes register memory. The sorting tree includes the following steps. At step 1, when a new DWT coefficient is ready, the first node in memory is loaded. If the tree is empty, the root node is created at the first address in memory, otherwise step 2 is executed. At step 2, if the new coefficient amplitude is greater than that of the currently loaded node, the number of greater coefficients is incremented and the right node is loaded, otherwise the left node is loaded. Step 2 is repeated until a NULL pointer address is reached. Then, a leaf node is created at the next available memory address, and the pointer of the node currently loaded (right or left) is updated with the new node address. Before overwriting the new node, the former coefficient value is sent to the next compression stage, as to perform sorting and compression in parallel. If the last coefficient is reached, step 3 is executed, otherwise step (i) is executed. At step 3, the circuit iterates on the nodes to find Cₘₐₓ and C_{N}, i.e., those having the maximum and N−1 greater coefficients (see the right most portion of FIG. 10) respectively. Meanwhile, the node pointers are reset with a NULL address. The sorting tree has step 4, where Cₘₐₓ and C_{N} are forwarded to the next compression stage, after which the sorting tree goes back to step 1. This sorting tree embodiment is convenient as it exhibits a complexity of O(n·log (n)), an improvement compared with O(n²) in existing alternatives, and uses two times less memory.

During the compression stage, the coefficients greater or equal to C_{N} are retained, normalized, and quantized from 16 to 6 bits, while the other coefficients may be discarded. The position of the retained coefficients are stored inside a 48-bits array. A coefficient normalization is performed by dividing the retained coefficients by Cₘₐₓ, while a requantization is performed by keeping the 6 MSB of the division. The 16 bit per 16 bit division is performed with a sequential non-restoring division circuit, which yields a simplified implementation. Only Cₘₐₓ is kept on 16 bits for allowing a resolution equivalent to the original neural data in the AP reconstruction.

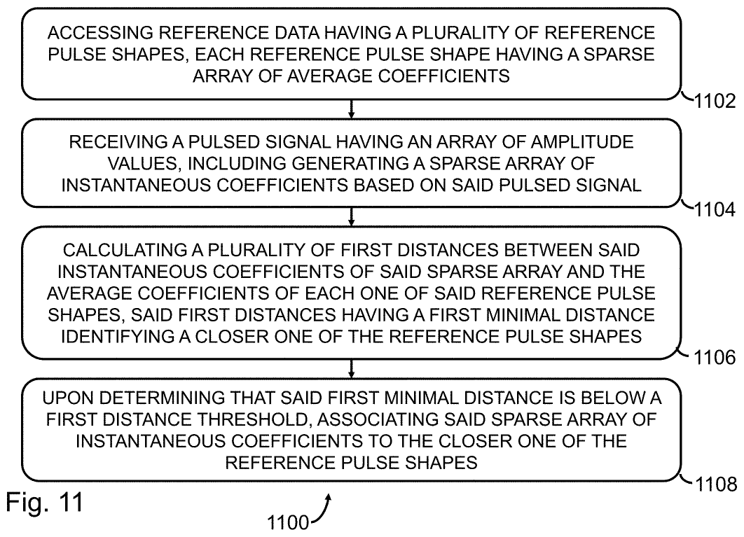

Once the detected AP are compressed, the pulse processing device can classify the detected AP into one of a plurality of reference shapes. FIG. 11 shows a flow chart of a method 1100 of associating a pulsed signal to a corresponding reference pulse shape. The method 1100 may be performed after method 900 described above with reference to FIG. 9. As shown, the method 1100 has a plurality of method steps which can be hardcoded in a printed circuit board in some embodiments.

At step 1102, reference data are accessed. The reference data typically have a plurality of reference pulse shapes S1, S2, . . . S_{B}, with each reference pulse shape having a sparse array of average coefficients [C̅₁, C̅₂, . . . , C̅_{N}]. As mentioned above, the coefficients may be wavelet coefficients in at least some embodiments.

At step 1104, a pulsed signal is received. The pulsed signal typically has an array of amplitude values [A₁, A₂, . . . , A_{M}], such as discussed above. However, in this method, step 1104 includes the generation of a sparse array of instantaneous coefficients [C₁, C₂, . . . , C_{N}] based on the pulsed signal. As can be understood, in at least some embodiments, the generation of the sparse array may be performed using the method 900 describe above. The instantaneous coefficients may thus be wavelet coefficients at least in some embodiments.

It is noted that the sparse array of instantaneous coefficients can be generated by performing one or more discrete transforms on the amplitude values of the pulsed signal. As such, the number N of instantaneous coefficients is smaller than the number M of amplitude values. It is noted that the number N of average coefficients corresponds to the number N of instantaneous coefficients. As such, the average coefficients and the instantaneous coefficients may be obtained using similar same discrete transforms. For instance, in embodiments where the discrete transform(s) is(are) discrete wavelet transform(s), the instantaneous coefficients are in fact instantaneous wavelet coefficients. An example of a discrete wavelet transform that was found to be satisfactory is the Symmlet-2 discrete wavelet transform. In these types of discrete wavelet transforms, the level 4 Symmlet-2 discrete wavelet transform was found convenient in at least some embodiments.

At step 1106, a plurality of first distances are calculated. Each first distance corresponds to the geometrical distance between the instantaneous coefficients of the sparse array and the average coefficients of a corresponding one of the reference pulse shapes. In other words, geometrical distances separating the sparse array from each one of the reference pulse shapes are calculated. Accordingly, if in some embodiments the reference data incorporates a number B of reference pulse shapes, a number B of distance distances may be calculated in this step. By calculating the first distances, it will become apparent that a minimal one of the first distances identifies which of the reference pulse shapes is closer to the sparse array.

At step 1108, the sparse array of instantaneous coefficients is associated with the closed one of the reference pulse shapes upon determining that the first minimal distance is below a first distance threshold.

Otherwise, the method 1100 can have a step of registering the sparse array of instantaneous coefficients as a sparse array of average coefficients of a new reference pulse shape in the reference data upon determining that the first minimal distance exceeds the first distance threshold.

In some embodiments, the first distances are L1 distances which are calculated by a relation equivalent to the following equation:

d_{L1}=Σᵢ₌₁^{N}|xᵢ−x̅ᵢ̅|, (1)

wherein i denotes an integer, N denotes a number of coefficients of the sparse arrays, xᵢ denotes the i^{th} instantaneous coefficients of the sparse array and x̅ᵢ̅ denotes the i^{th} average coefficients of a corresponding one of the reference pulse shapes. However, in some other embodiments, the Euclidian distance may also be used, to the cost of additional computational requirements as Euclidian distance requires additional arithmetic operations (e.g., difference of squares, square-roots).

In some embodiments, the method 1100 includes a step of updating the average coefficients of the closer one of the reference pulse shapes on the basis of the instantaneous coefficients of the sparse array. By updating the average coefficients of a given one of the reference pulse shapes, the updated reference pulse shape may drift away from the original reference pulse shape. As such, the method 1100 can include a verification step comprising the calculation of second distances between the updated average coefficients of the closer one of the reference pulse shapes and the average coefficients of the other ones of the reference pulse shapes. As for the first distances, the second distances will typically have a second minimal distance identifying a closer one of the other one of the reference pulse shapes. In these embodiments, the method 1100 can include a step of registering the instantaneous coefficients of the sparse array as a sparse array of average coefficients of a new reference pulse shape in the reference data upon determining that the second minimal distance is greater than a second distance threshold.

It is envisaged that the controller incorporating the pulse classification module, or the pulse classification module itself, can have a footprint below 5 cm³, below 2.5 cm³ and most preferably below 1 cm³, with a power consumption below 1 mW, preferably below 500 μW and most preferably below 100 μW.

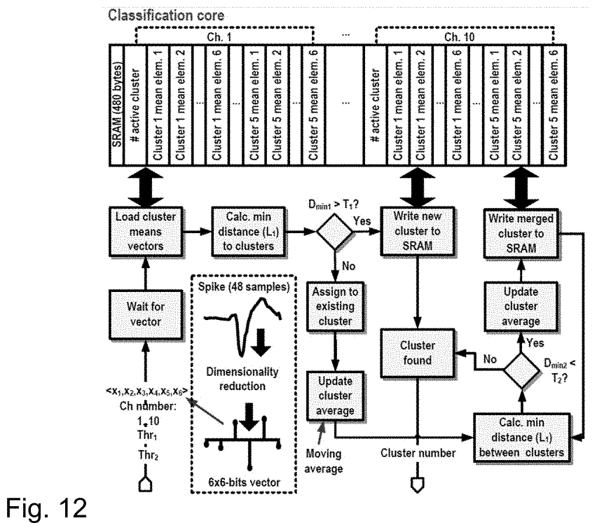

The following paragraphs describe a specific example implementation of such a method. As discussed above, a major burden for AP classification is the dimensionality reduction step, which consumes time, power, and area. Fortunately, the aforementioned compression module produces a dimensionality reduction at each of the DWT levels. The proposed strategy consists generating a 6×6-bit element array per AP using the level-3 (L3) compressed DWT coefficients. This type of strategy can use ˜3 times less memory compared to the non-compressed coefficients, while negligibly impacting the results (−1.2% classification results, worst simulated case). A block diagram implementing an example of that method is depicted in FIG. 12. When a new array (V_{L3}) become available, the average array of each cluster is loaded from SRAM. Then, the L₁ distance is computed between V_{L3} and the cluster average values. If the minimal distance (Dₘᵢₙ₁) is greater than the threshold T₁, a new cluster is created and initialized with V_{L3}. Otherwise, V_{L3} is associated to the closest cluster, and its average array is updated using a moving average calculation. In the latter case, the L₁ distance is computed between the updated cluster average array and the other clusters average arrays. If the minimal distance (Dₘᵢₙ₂) is less than the threshold T₂, the clusters are merged together. To calculate the thresholds T₁ and T₂, we can model the detected waveform fed to the compression module as comprising an AP component and a noisy component:

V_{fed}=V_{AP}+V_{Noise}, (2)

where V_{AP} is a noiseless AP waveform, and V_{Noise} represents an additive noise array. The Symmlet-2 DWT has a major advantage of being an orthonormal transformation. A property of such a transformation is that a Gaussian white noise remains Gaussian under an orthogonal transformation. Thus, if V_{Noise} is fed at the input of the Symmlet-2 DWT module, each level will produce a noise array with a scaled standard-deviation compared to the original noisy array. The Symmlet-2 DWT of FIG. 10 can be expressed in terms of orthogonal filter convolutions, namely with the filters H(z) and G(z) producing the approximative and detailed coefficients respectively. The L3 output array (V_{L3}(z)) of the DWT is expressed by:

V_{L3}(z)=[G(z)\textcircled{*}[H(z)\textcircled{*}[H(z)\textcircled{*}V_{fed}(z)]↓2]↓2]↓2, (3)

where ↓2 represents a downsampling by 2 operation, \textcircled{*} is a convolution operation, and the filters H(z) and G(z) are expressed by:

H(z)=H₅[H₂+(H₁H₂−1)z⁻¹+(1−H₃)z⁻²+H₁(1−H₃)z⁻³]

G(z)=H₆[−H₂+(1−H₁H₂)z⁻¹+H₃z⁻²+H₁H₃z⁻³], (4)

where H₁-H₆ are the filter coefficients and scaling factors of the Symm-2 DWT. Computing the amplitude of these filters at π/2 can find the gain at each DWT level:

$$\vert H ( \frac{π}{2} ) \vert \approx 1.13 and \vert G ( \frac{π}{2} ) \vert = 1 ,$$

meaning that if the DWT input is V_{Noise}, the L3 output array (V_{L3,N}) will also be Gaussian with an amplitude gain of 1.28, and a standard deviation 1.28 times greater than V_{Noise}. Thanks to the adaptive threshold scheme provided in the AP detector module, we already computed σₐ of |V_{Noise}|, which relates to the standard deviation of V_{Noise} by the scaling factor \sqrt{1−2/π}. Thus, an approximated value of the standard deviation of the L3 coefficient (σ_{L3,N}) is already available according to V_{Noise}.

The calculation of the L₁ distance between the cluster average array and V_{L3} is influenced by the noise in V_{fed}, which is reflected as additive noise inside V_{L3} as stated above. This bias error can be modeled by summing the absolute value of the components of V_{L3,N}. Since the components of V_{L3,N} are assumed to be independent, the standard deviation of the bias error (σₑ), i.e. the σ of Σₙ₌₀⁵|V_{L3,N}(n)|, is equal to \sqrt{1−2/π}·\sqrt{6}·σ_{L3,N}. Since σ_{L3,N} is already known, we set T₁ and T₂ as multiple of σₑ, the value of which is:

σₑ=\sqrt{1−2/π}·\sqrt{6}·σ_{L3,N}=1.28·\sqrt{6}·σₐ≈3.14·σₐ, (5)

as to reduce the classification errors due to the signal noise.

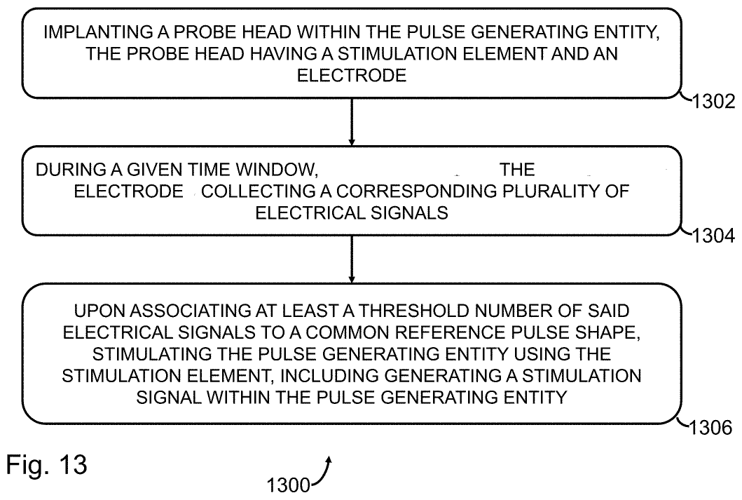

Once the detected AP are compressed and classified, the result may be used to stimulate the pulse generating entity on the go. For instance, FIG. 13 shows a flow chart of a method 1300 of stimulating a pulse generating entity such as brain tissue, heart tissue and the like. The method 1300 may be performed after methods 900 and 1100 described above with reference to FIGS. 9 and 11. As shown, the method 1300 has a plurality of method steps which can be hardcoded in a printed circuit board in some embodiments.

At step 1302, a probe head is implanted within the pulse generating entity. As discussed above, the probe head can be part of a probe unit communicatively coupled to the controller. The probe head typically has one or more stimulation elements and one or more electrodes. In some embodiments, the stimulation element includes an optical fiber delivering an optical stimulation signal within the pulse generating entity. In some other embodiments, the stimulation element can include an electrode delivering an electrical stimulation signal within the pulse generating entity. The probe head may also incorporate one or more optical fibers which is configured to collect optical signals from the pulse generating entity, when such pulses are optical. In these embodiments, the electrode(s) may be omitted.

At step 1304, the electrode collects a plurality of electrical signals during a given time window. Depending on the embodiment, the time window can extend during at least 100 ms, preferably at least 50 ms and most preferably at least 25 ms.

At step 1306, upon associating at least a threshold number of the electrical signals to a common reference pulse shape, for instance using the method 1100 of FIG. 11, the pulse generating entity is stimulated by the stimulation element. The common reference pulse shape can be selected from a number of predetermined reference pulse shapes, for instance. The stimulation of the pulse generating entity by the stimulation element includes the generation of a stimulation signal therewithin. In some embodiments, the threshold number is at least 2, preferably at least 3, and most preferable at least 5. However, the threshold number can be more than 5.

Accordingly, as the pulse generating entity is stimulated with the same stimulation signal over and over again whenever a few electrical signals of the same types are being detected within the same time window, the behaviour of the pulse generating element can be conditioned. In some embodiments, the rat's behaviour may be rewarded or punished with a given stimulation signal each time it is detected that the rat thinks or does something that triggers the firing of the neuron(s) surrounding the electrodes of the probe unit. As such, the stimulation signal can be associated with an expected reaction of the pulse generating entity, so that the pulse stimulation module can condition the pulse generating entity to react according to the expected reaction upon a corresponding stimulation.

As such, in some embodiments, the stimulation signal may be selected on the basis of the detected common reference pulse shape. For instance, a first stimulation signal may be used when the detected common reference pulse shape is identified to be a first reference pulse shape, a second stimulation signal may be used when the detected common reference pulse shape is identified to be a second reference pulse shape, and so forth. In these embodiments, the first and second stimulation signals may differ from one another, in order to properly condition the pulse generating entity. Differences in these stimulation signals can be provided in terms of amplitude, frequency, pulse shape and/or color when the stimulation signals are optical stimulation signals.

In some embodiments, the given time window can be modified on the go. The given time window can even be slid over the duration of a received raw signal, with the compression and classification modules continuously compressing and classifying any AP detected within the given time window at all times, in some embodiments.

It is envisaged that the controller incorporating the pulse stimulation module, or the pulse stimulation module itself, can have a footprint below 5 cm³, below 2.5 cm³ and most preferably below 1 cm³, with a power consumption below 1 mW, preferably below 500 μW and most preferably below 100 μW.

A CL link can be established between the stimulator and the neural recording circuit of the mixed-signal SoC through the ND-IC. The FPGA is counting the number of detected APs in real time within a sliding time window and with respect to their respective cluster associations (i.e. classification) established by the classification module inside the ND-IC. Each AP-to-cluster association can be set to trigger a stimulation pattern after a predetermined amount of occurrences found within a sliding time window. The duration of the sliding window can be configured to target numerous recognizable firing patterns, like AP bursts of varying lengths, while the stimulation patterns, i.e. number of pulses, pulse width, and the stimulation channel, are also configurable.

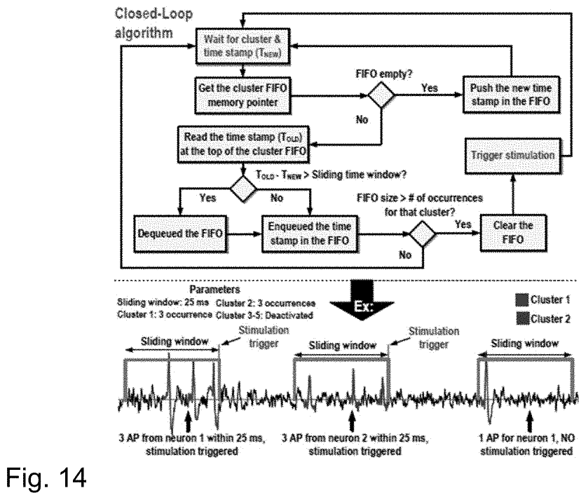

The CL scheme, whose diagram is shown in FIG. 14, is optimized to use only basic FIFO structures implemented inside the low-power FPGA. Each cluster pertaining to each channel is associated with a unique FIFO in SRAM memory, which contains the latest AP timestamps for that cluster. When a new AP-to-cluster association becomes available, a memory pointer to the FIFO structure belonging to that cluster is retrieved. If the FIFO is empty, the AP timestamp (T_{NEW}) gets enqueued. Otherwise, the module reads the older timestamp (T_{OLD}) at the top of the FIFO. If T_{OLD}−T_{NEW} is greater than the sliding window length, the top of the FIFO dequeued. Then, T_{NEW} is enqueued, and if the FIFO size is equal to the number of APs required to trigger a feedback stimulation pattern, the FIFO is cleared and a trigger is issued.

Following the preliminary validation of the system, we performed CL optogenetic stimulation with a freely-moving mouse expressing ChR2-mCherry. In this experiment, the base station was placed beside the animal cage, approximately 1.5 feet away from the animal. The threshold gain was set to 4.2, the AP CR was set to 4.17, the maximum number of clusters to five, and the feedback stimulation was set to 1× pulse of 10 ms (20 mA flowing through the LED). The CL algorithm was configured to trigger when 6 APs belonging to cluster 1 were found within a 150-ms time window. This configuration was selected after manually looking at close successive APs belonging to the same cluster. This simple approach was used to validate the system in freely-moving animals. However, further analysis of the neural bursting activity may be required to find the optimal configuration.

As depicted in FIG. 12, the classification module includes a FSM and a 480-bytes SRAM memory block. This memory is separated in 10 segments of 32×12 bits each, which are used for storing up to five cluster averages and the number of active clusters (N_{active}) per channel. Upon the reception of a new V_{L3} array, the FSM computes the L₁ distance between V_{L3} and each cluster average. The cluster averages array components are using a 12-bit fixed-point notation, in which only the 6-MSB are used to compute the L₁ distance. If a new cluster is created (i.e. Dₘᵢₙ₁>T₁), N_{active} is incremented and V_{L3} is written in the SRAM at the N_{active}^{th} cluster average address. Otherwise, V_{L3} is associated to the closest cluster before its average array is updated using a cumulative moving average calculation. This is performed according to the following equation:

Cᵢ(n)=Cᵢ(n−1)+(V_{L3,i}(n)−Cᵢ(n−1))/2⁶, (6)

where Cᵢ is the i^{th} element of the cluster average array C and V_{L3,i} is the i^{th} element of the new array V_{L3}. Equation (5) is computed using only simple bit shifting and addition/subtraction operations. Then, the FSM computes the L₁ distance between the updated array C and all the other cluster average arrays. If two clusters get too close to each other (i.e. Dₘᵢₙ₂<T₂), they are merged together by taking the average of the two arrays. Then, N_{active} is decremented, and the FSM reorders the cluster averages stored in the SRAM. At worst, forty clock cycles are required to complete a classification step, which yields a minimal operating frequency of 40 kHz to handle 10 channels, assuming a maximal firing rate of 100 AP/s.

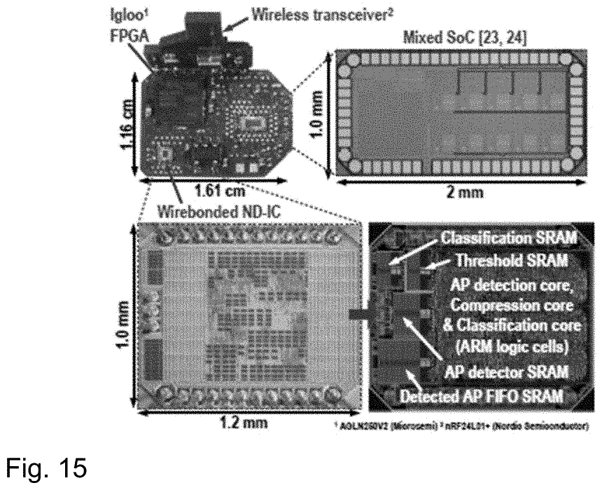

The integrated headstage and the ND-IC micrograph are shown in FIG. 15, along with the ND-IC layout, in which the circuits and SRAM blocks are identified. The SRAM occupies ˜15% and the digital circuits ˜50% of the 1.2 mm² ND-IC die area. The ND-IC is directly wirebonded on the headstage PCB along with the mixed-signal SoC to save space, next to the low-power FPGA. The headstage consists of two interconnected PCBs: i) the base PCB where the ICs are wirebonded, and ii) the PCB that holds the wireless transceiver placed at 90 degrees from the first PCB. The fully packaged system is shown in FIG. 4. A drop of epoxy is applied on the dies to protect the wirebonds, and a 40-mAh LiPo battery is placed above the electronic components to fill any unused space. The maximum temperature increase that was measured at the bottom of the optrode (FIG. 4) over the whole lifespan of the battery) is 0.9° C. This temperature increase is in the range of normal physiological conditions, and cannot harm the animal tissues. The packaged system with the battery weighs 3.0 g, and occupies 1.13 cm³.

The following paragraphs present simulation results of the proposed AP detector, and a comparison with other detection techniques. For these tests, we generated synthetic neuronal signals built using real, previously-recorded AP waveforms. These signals were designed to have a non-constant firing rate and a realistic SNR by adding both white Gaussian noise and neuronal noise. The SNR is measured as follows:

SNR=10·log(σ_{AP}²/(σₙₒᵢₛₑ²+σₙₑᵤᵣₒₙₐₗ²)), (7)

where σ_{AP}² is the mean variance of the APs, σₙₒᵢₛₑ² is the variance of the added neural noise and σₙₑᵤᵣₒₙₐₗ² is the mean variance of the neuronal noise.

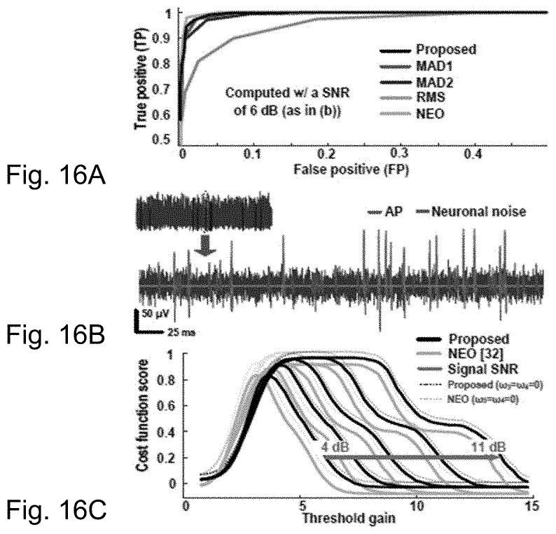

We compared the absolute value operator used with the MAD threshold (MAD1, δ=median{|x|}/0.6745), the MAD threshold windowed (MAD2), the RMS threshold windowed and the NEO operator with the MAD threshold. Both windowed algorithms are calculated over a window of 50 ms. FIG. 16A shows the receiver operating characteristics (ROC) computed with a signal with a standard SNR of 6 dB, as seen in FIG. 16B. As can be seen, the proposed algorithm performs better than RMS and MAD2, and has equivalent or better performance than MAD1. However, our algorithm is subtly overtaken by NEO. The trade-off between this slight ROC increase versus complexity for NEO needs to be further explored. For that purpose, we devised a simple cost-function (CF):

CF=ω₁·TP+ω₂·FP+ω₃·Aᵢ/Aⱼ+ω₄·Pⱼ/Pᵢ, (8)

where ω_{1,2,3,4} are the weight (1, 1, 0.025 and 0.025 respectively), Aᵢ and Pᵢ are the synthesized estimated circuit area and estimated dynamic power consumption, respectively, of the algorithm being tested, while Aⱼ and Pⱼ represent the same parameters for the other algorithm. Post-synthesis power and area estimations (using Synopsis) give 30 nW (F_{clk}=20 kHz) and 0.013 mm² respectively for the proposed algorithm, while similar estimations using a 0.13-m process yield 50 nW (F_{clk}=20 kHz) and 0.021 mm². Higher area and power consumption for NEO were predictable, since it requires a minimum of two extra multiplications. The CF results are presented in FIG. 16C for an SNR ranging from 4 to 11 dB. As seen, both algorithms yielded the same CF results for an SNR of 4-dB, while the proposed algorithm scored higher for the other SNRs. The 4-dB CF is due to NEO performing well with extremely noisy signals, but since both detectors produce similar detection performances under more realistic SNRs, the NEO CF is smaller due to a higher complexity.

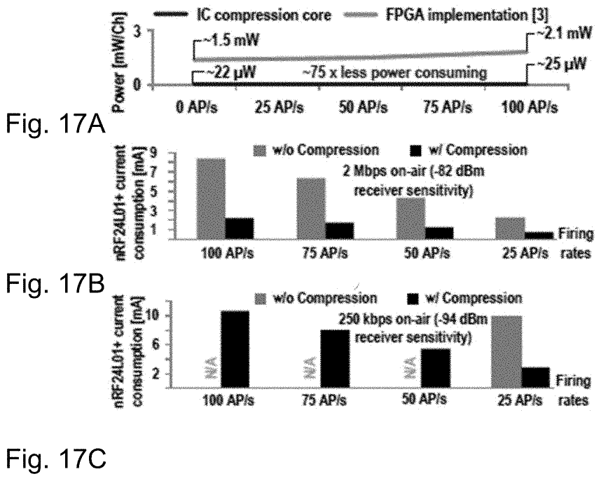

A signal-to-noise distortion ratio (SNDR) analysis using in vivo collected AP waveforms is provided. FIG. 17A shows the power consumption measurements of the proposed compression module versus existing alternatives. On average, the proposed IC compression module consumes ˜75× less power, justifying the integrated approach. FIGS. 17B and 17C show the current consumption of the wireless transceiver with and without compression (CR of 4.17) at 2 Mbps and 250 kbps on-air datarates respectively. As can be seen, compression significantly reduces the power consumption (by up to 3.8×) and increases the range by allowing lower datarates. At 250 kbps, receiver sensitivity is improved by −12 dBm (twice the range), which would not have been possible without compression at firing rates higher than 25 AP/s.

The following presents the measured results of the proposed classification algorithm when compared to other sorting techniques. For the first tests, we used readily available synthetic neural signals built using real AP waveforms. This public neural data bank is used to benchmark the Waveclust off-line neural sorting software, making it perfectly suited to benchmark our algorithm. We used 20 datasets, each of which encompass, on average, 3,400 different AP waveforms belonging to up to three clusters per set. Most of the datasets include overlapping AP, adding an extra level of difficulty.

The proposed dimensionality reduction technique is compared with 1) the more computationally-intensive principal component analysis (PCA) (w/ three components) as a reference (used as ground truth), 2) with the discrete derivative (DD) (using 10 coefficients found with the Lillifor test for normality), and 3) with a DWT algorithm based on the Haar mother wavelet (which is the simplest and most popular mother wavelet). The dimensionality reduction for the Symmlet-2 and for the Haar DWT are validated using the L3 and L4 coefficients. We used the same K-means classification algorithm to benchmark all the dimensionality reduction techniques.

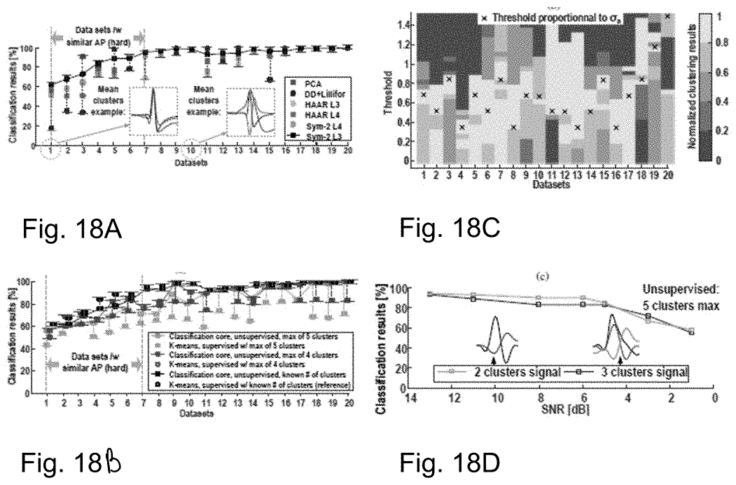

FIG. 18A shows the classification results. The black curve shows the proposed Symmlet-2 results using the L3 coefficients, while the other results are presented by a coloured square. As can be observed, the proposed technique yielded one of the best results for almost all datasets, equal to or just a little behind the PCA. Overall, the third best results are generated by the Symmlet-2 (L4), followed by the Haar (L4), the Haar (L3), while the worst results are generated by the DD algorithm. These results demonstrate that our proposed dimensionality reduction technique can be superior to any existing approach, while generating similar results to those of a complex PCA.

FIG. 18B shows the on-line unsupervised classification results of the proposed algorithm (solid lines) compared to the ones obtained with K-means (squares). In this simulation, the maximum number of clusters is limited to: 1) the known number of clusters (black), 2) a maximum of four clusters (red) and 3) a maximum of five clusters (green). As can be seen, when the number of clusters is known, the proposed algorithm behaves almost as well as K-means, while being 100% unsupervised. When the maximum number of clusters is set to four and five, the proposed technique still produces the accurate number of active clusters, and the results remain highly competitive. This contrasts with K-means, with which the results degenerate significantly when the number of clusters is overestimated.

FIG. 18C shows the normalized classification performance according to the threshold T₁ (T₂ is set to ˜0.6×T₁). The yellow area for each dataset indicates the region in which the threshold produces the optimal classification results, while the black X indicates the threshold value that was computed automatically using σₐ. We can see that the black Xs are either close to, or inside, the optimal threshold region for almost all datasets. Indeed, the calculated threshold lies in the optimal region 80% of the time, thus promising a reliable performance in real life.

FIG. 18D shows the unsupervised classification performances according to the signal SNR, for two neural signals encompassing AP waveforms from two and three clusters respectively, and for a maximum number of five clusters. The classification results are almost constant, and start dropping under 5 dB. Even with a low SNR of 3 dB, the classification results maintain a success rate of approximately 70%.

The power breakdown of the ND-IC is depicted in FIG. 19A. The detection, compression and classification modules consume 5.8 W, 22.6 W and 3.0 W per channel, respectively. These measurements include the SPI modules and the communication FSMs. The power consumption of the ND-IC according to the AP firing rate is shown in FIG. 19B. The power consumption baseline is 56.4 W/Ch, and increases proportionally to the firing rate with a slope of 0.01 W/AP. The AP-power relation is due to the deactivation of some circuits when no AP is being detected, compressed or sorted. Finally, the power consumption of the ND-IC according to different module operating frequencies is shown in FIG. 19B.

The headstage was validated in vivo within two experiments, the first involving an anesthetized Long-Evans rat, and the second a freely-moving mouse. The first experiment demonstrated the system's ability to detect, compress, sort, and generate CL trigger signals (without feedback optical stimulation) in vivo. Following good preliminary results, the second experiment demonstrated the CL operation of the headstage with a freely-moving mouse.

For the preliminary in vivo trials, a Long-Evans rat was anesthetized with ketamine/xylazine solution. A glass micropipette (5-μ tip) filled with saline solution was lowered in the LD brain region of the thalamus (4.5-5 mm depth). For the freely-moving in vivo trials, a B6SJLF1/J mouse (Jackson Laboratory, USA) was injected with 300 nL of AAV5-mDlx-ChR2-mCherry-Fishell-3 (Plateforme d'Outils Moléulaires, Canada; titre 2.0E13 GC/mL), in the PL region of the medial prefrontal cortex (1.80 AP; 0.30 ML and −1.70 DV from Bregma) followed by an implantation of a detachable optrode (Doric Lenses, Canada), consisting of one optical fiber (200-m module; 0.66 NA) and four Tungsten microelectrodes (50-m flat tip). It should be noted that the ChR2-mCherry stimulation will result in the activation of the interneurons, an inhibitory effect is therefore expected. The recordings were carried out seven months after the viral injection, so the animal was able to fully recover from the surgical procedure and to adopt a normal behaviour with the headstage in place. All protocols were performed in accordance with guidelines from the Canadian Council on Animal Care.

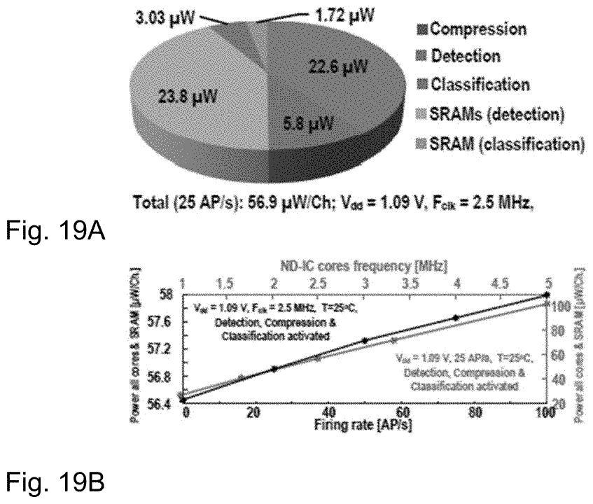

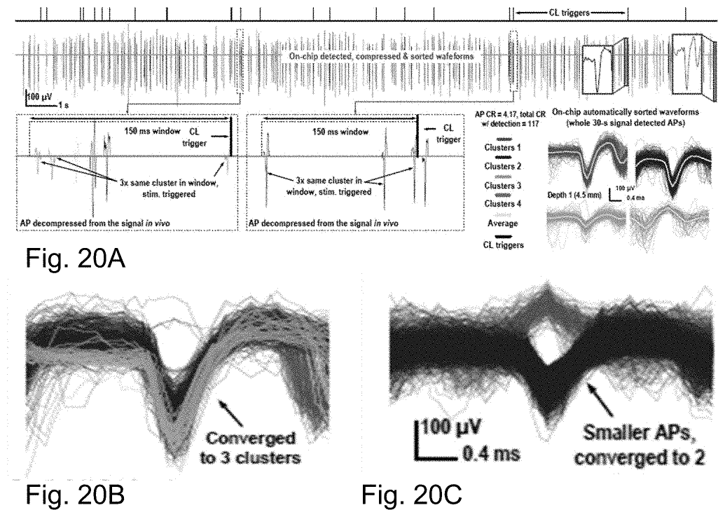

In these experiments, the threshold gain was set to 3.5, the AP CR was set to 4.17 and the maximum number of clusters was set to five. As shown in FIG. 20A, the ND-IC enclosed within the wireless headstage was able to automatically detect, compress, and sort the AP on-the-fly. The overall CR achieved with detection and AP compression was 117. The detected clusters were used to generate a trigger signal in situ (black signal in FIG. 20A) to validate the CL scheme running in real time. The trigger was configured to occur if three AP per clusters were found within a 150-ms time window. FIGS. 20B and 20C show the automatic classification results at two different brain depths. FIGS. 20A, 20B and 20C show that the classification algorithm accurately sorted most of the APs, since each cluster encompasses a different AP waveform. We also see that the algorithm converged to an appropriate number of clusters.

Following the preliminary validation of the system, we performed CL optogenetic stimulation with a freely-moving mouse expressing ChR2-mCherry. In this experiment, the base station was placed beside the animal cage, approximately 1.5 feet away from the animal. The threshold gain was set to 4.2, the AP CR was set to 4.1, the maximum number of clusters to five, and the feedback stimulation was set to 1× pulse of 10 ms (20 mA flowing through the LED). The CL algorithm was configured to trigger when 6 APs belonging to cluster 1 were found within a 150-ms time window. This configuration was selected after manually looking at close successive APs belonging to the same cluster. This simple approach was used to validate the system in freely-moving animals. However, further analysis of the neural bursting activity may be required to find the optimal configuration.

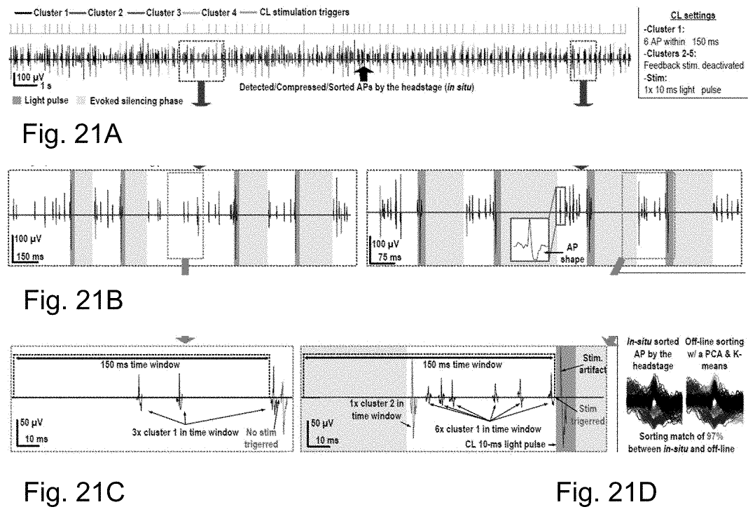

FIG. 21A shows a reconstructed signal of 30 s after decompression, along with some generated CL stimulation triggers (blue signal). The CL stimulation signal was of ˜2.5 pulses/s on average, and the overall CR was of ˜46 in this experiment. FIG. 21B shows two signal closeups in which the stimulation pulses and the evoked silencing phases, due to the inhibitory neuron activation, are visible. FIG. 21C shows two enlargements zooming in on specific AP trains. The first enlargement shows three APs belonging to cluster 1 within the 150-ms time window, thus triggering no stimulation, while the other enlargement shows 6 APs belonging to cluster 1 within the time window, followed by a stimulation pulse and a silencing phase. These results show that the proposed system works well within an in vivo experimental setting with a freely moving mouse, since it was able to induce silencing phases by stimulation, as expected.

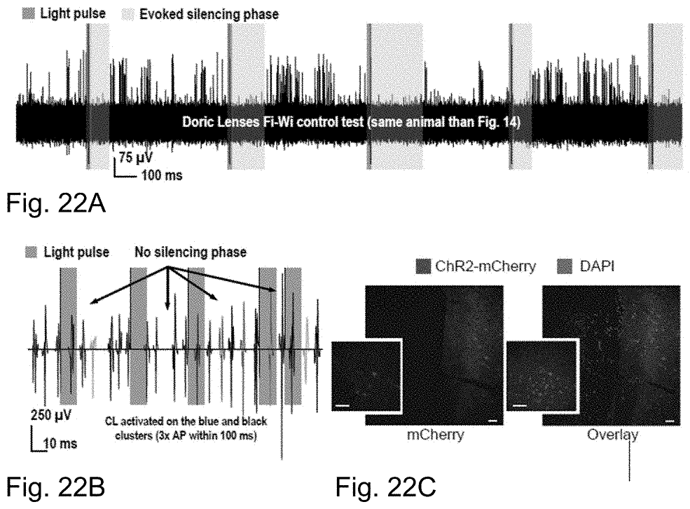

After this experiment, three validation experiments were performed. The first validation involved the same animal and a commercial Fi-Wi optogenetic system from Doric Lenses, Canada. FIG. 22A shows a sample signal recorded using the Fi-Wi system during stimulation. We can see that silencing phases are also observed with the commercial system after each stimulation light pulse. FIG. 22B shows CL stimulation performed in a non-injected mouse (no ChR2 expression) using our headstage. No silencing phases are visible upon stimulation. FIG. 22C shows a confocal image of mPFC sections from the mouse used in the in vivo experiments 11 months post viral injection. The cells expressing the ChR2-mCherry (red) construct and the nuclei labeled with DAPI (blue) are visible, demonstrating that the ChR2 was expressed at the time of the experiment. These validation experiments show that the silencing phases shown in FIGS. 21A, 21B and 21C were most likely caused by inhibitory neuron activation.

Stimulation artifacts are visible in FIGS. 21A, 21B and 21C. To reduce artifacts, we used blunt (opaque polyimide covered) electrodes to prevent the direct light reaching the tip of the electrode, while the electrode tips were placed in an area with little light. However, small artifacts (caused by light or other sources) are still being detected. Assuming the optrode stays still, we expect these artifacts to have similar shapes over time. Our ND-IC, which performs on-line waveform sorting, has an important advantage to prevent noise artifacts compared to other solutions: it can process the light artifact just as any other waveshapes and sort them into a separated cluster to isolate them from the neural waveforms, similarly to the off-line strategy proposed in [38] also intended to eliminate the stimulation artifacts. The ND-IC action is illustrated in FIG. 21D, where most of the artifacts were sorted within the blue cluster. It is then possible to deactivate this cluster and prevent triggering the CL stimulation upon the artifacts.

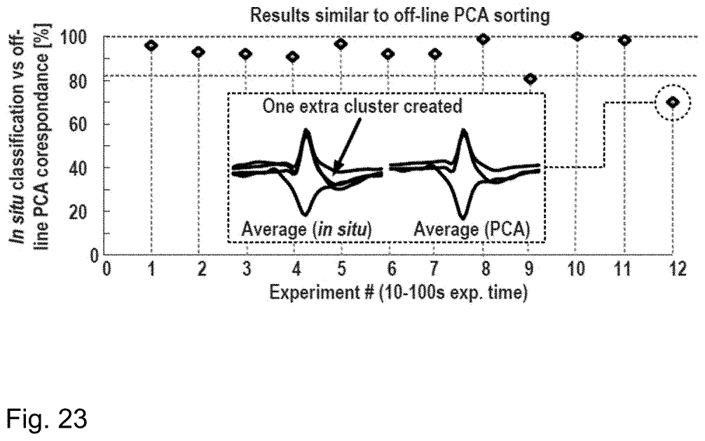

The AP automatically sorted by the ND-IC are shown compared to the same AP sorted off-line by a PCA and K-means in FIG. 21D. We report a classification match of 97% between the AP sorted live by the ND-IC and off-line by the PCA. To take this analysis further, FIG. 23 shows the classification matching between the sorted AP in situ by the headstage vs the off-line sorted AP using a PCA and K-means for 12 freely-moving experiments. Eleven of the twelve experiments generated a match of more than 80%, while only one produced a matching of ˜70%. This lower classification matching can come from the fact that, for the same dataset, our technique have converged to an extra cluster compared to the preconfigured number of cluster used with PCA and K-means. Given that the number of clusters is not known a priori for the ND-IC, the proposed DWT approach can trigger the creation of up to 5 clusters when it judge that the waveforms are slightly different, while it must be known a priori for the PCA and K-means. Our classifier has the advantage of using unsupervised classification without training while the PCA and K-means require the whole dataset at once. Since any additional clusters can catch a certain number of APs, classification mismatches can then occur between both classifiers. If judged necessary, it is possible to increase the accuracy of our classifier by entering the maximum number of clusters manually after data inspection, or by running a training phase on a subset of the dataset to select optimally what DWT coefficients to use in the sorting array. This procedure would most likely increase the sorting accuracy.

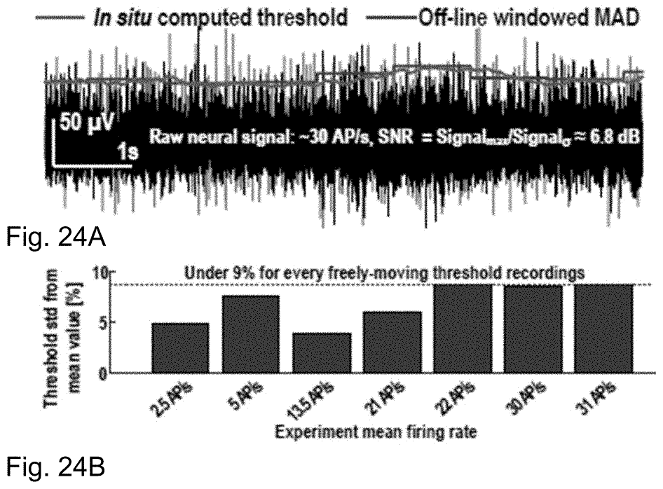

We tested the adaptive threshold algorithm in vivo by simultaneously transmitting the threshold calculated inside the headstage to the base station along with the raw recorded signal (SNR ≈6.8 dB). FIG. 24A shows the threshold calculated by the headstage (in blue), the threshold calculated using the MAD windowed operator (off-line, in red), both with a Gₜₕᵣ of 4.2. The proposed threshold is almost perfectly superimposed over the threshold calculated by the MAD operator, showing that the σ estimation and the gains are accurately computed. Since σₐ is based on a signal containing APs, it is expected that the firing rate slightly impacts the computed threshold. FIG. 24A shows the threshold standard deviation from its mean value for seven freely-moving experiments, having an average firing rate from ˜2.5 AP/s to ˜31 AP/s. We can see that the threshold varies from ˜5% for low firing rates, and up to ˜8% for higher firing rates, thus having a low impact on the calculated threshold value. Simulation with a synthetic signal having a realistic SNR of 6.8 dB (i.e. similar to the in vivo waveforms of FIG. 24A) shows that the AP firing rates can induce a variation of around 0.22% per AP/s in the calculated threshold. Thus, this corresponds to a variation of 2.2% for a typical firing rate of 10 AP/s, while it corresponds to a variation of 22% for a firing rate of 100 AP/s, which is less likely to occur.

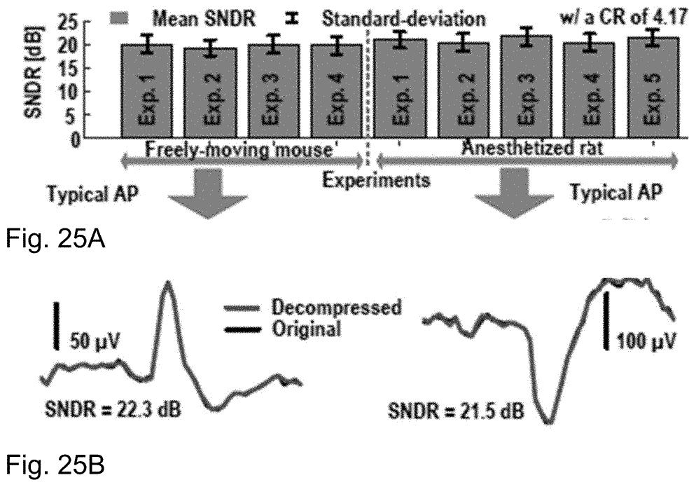

FIG. 25A shows the mean compressed AP SNDR, acquired during four experiments with the freely-moving mouse and five experiments with the anesthetized rat. The SNDR is defined by: SNDR=20 log(||x||₂/(||\hat{x}−x||₂)), where x is the original AP and \hat{x} is the reconstructed waveform. We can see in FIG. 25A that the mean SNDR is kept constant around 20 dB for all the experiments involving the two animals with different AP waveforms. FIG. 25B presents two typical compressed APs collected within the brain of the mouse and the brain of the rat, and superimposed on their non-compressed counterpart, showing they are visually indistinguishable

The proposed headstage and ND-IC are compared to recently published systems and experimental settings for either CL optogenetics, optogenetics with neural recording or CL electrical stimulation. As illustrated, the proposed wireless headstage has features comparable to complex wired PC-based CL optogenetics settings. The headstage calculates its CL optogenetic triggers based on the AP classification, and also on the AP bursting, which are two essential features for performing neural decoding. This contrasts with simple trigger solutions based on AP detection, PID controller based on the LFP energy or open-loop optogenetic wireless systems. The proposed ND-IC integrates more features (detection, compression, and classification) than the other reported systems. Most state-of-the-art CL optogenetic systems are based on simplistic feedback algorithms, and do not transmit the neural data wirelessly for providing live or post-experimental analysis. In contrast, the proposed IC is the only solution that performs AP detection and AP compression to further reduce the amount of data to transmit, which can monitor all the 10 neural channels simultaneously. Moreover, the proposed headstage has the lowest reported weight with the battery (3.0 g), which is critical to enable experiments with small freely-moving rodents. Conversely, recent publications only present ICs that are not integrated within fully working systems, and only tested in vivo with anesthetized animals. To the best of our knowledge, we have reported the first integrated circuit for CL optogenetics demonstrated in vivo within freely-moving animal trials. Future works will focus on integrating the ND-IC, the SoC, the CL algorithm and a controller to interface with the external wireless transceiver in the same CMOS chip, to reduce further the size and power consumption.

We presented a miniature wireless electro-optic headstage that is the first attempt towards complex CL optogenetics in the brain of freely-moving rodents. The headstage is based on a custom 0.13-m ND-IC than can detect, compress, and sort the neural signals on-the-fly over 10 channels in parallel without supervision, enabling a CL optogenetic strategy in situ. The ND-IC can detect the APs using an adaptive threshold, compress the APs using a DWT-based algorithm, and automatically classify the APs by reusing the sub-products of the compression and detector modules. The headstage can trigger complex CL optical stimulation patterns using the AP classification information along with their time occurrence within a sliding time window. Measurement and simulation results show that the ND-IC can detect, compress, and sort the AP with remarkable accuracy. The electro-optic headstage and the ND-IC have been validated in vivo in the brain of a rat and of a freely-moving mouse expressing ChR2-mCherry. The results show that our system works well within an in vivo experimental setting with a freely moving mouse.

As can be understood, the examples described above and illustrated are intended to be exemplary only. The scope is indicated by the appended claims.

What is claimed is:

1. A method of associating a pulsed signal to a corresponding reference pulse shape, the method comprising:
accessing reference data having a plurality of reference pulse shapes, each reference pulse shape having a sparse array of average coefficients;
receiving a pulsed signal having an array of amplitude values, including generating a sparse array of instantaneous coefficients based on said pulsed signal;
calculating a plurality of first distances between said instantaneous coefficients of said sparse array and the average coefficients of each one of said reference pulse shapes, said first distances having a first minimal distance identifying a closer one of the reference pulse shapes; and
upon determining that said first minimal distance is below a first distance threshold, associating said sparse array of instantaneous coefficients to the closer one of the reference pulse shapes.

2. The method of claim 1 further comprising, upon determining that said first minimal distance exceeds said first distance threshold, registering the sparse array of instantaneous coefficients as a sparse array of average coefficients of a new reference pulse shape in the reference data.

3. The method of claim 1 wherein at least one of said first distances is a L1 distance given by a relation equivalent to the following equation:
d_{L1}=Σᵢ₌₁^{N}|xᵢ−x̅ᵢ̅|,
wherein i denotes an integer, N denotes a number of coefficients of said sparse arrays, xᵢ denotes the i^{th} instantaneous coefficients of said sparse array and x̅ᵢ̅ denotes the i^{th} average coefficients of a corresponding one of the reference pulse shapes.

4. The method of claim 1 further comprising updating said average coefficients of the closer one of the reference pulse shapes based on said instantaneous coefficients.

5. The method of claim 4 further comprising calculating a plurality of second distances between the updated average coefficients of the closer one of the reference pulse shapes and the average coefficients of the other ones of the reference pulse shapes, the second distances having a second minimal distance identifying a closer one of said other ones of the reference pulse shapes.

6. The method of claim 5 further comprising, upon determining that said second minimal distance is greater a second distance threshold, registering the instantaneous coefficients as a sparse array of average coefficients of a new reference pulse shape in the reference data.

7. The method of claim 1 wherein said generating said sparse array of instantaneous coefficients includes performing one or more discrete transforms on said amplitude values of said pulsed signal.

8. The method of claim 7 wherein said performing includes performing one or more discrete wavelet transforms on said amplitude values of said pulsed signal, said instantaneous and said average coefficients being wavelet coefficients.

9. The method of claim 1 wherein said pulsed signal is generated by at least one of brain tissue and a heart tissue.

10. A method of sorting coefficients associated to a pulsed signal, the method comprising:
sampling a pulsed signal, including generating an array of amplitude values;
performing a discrete transform using said amplitude values of said array, including generating an array of coefficients indicative of an energy distribution of said pulsed signal, wherein a number of said coefficients is smaller than a number of said amplitude values; and
sorting said coefficients using a tree sorting algorithm, said tree sorting algorithm finding a maximal one of said coefficients and an N^{th} maximal one of said coefficients, including generating a sparse array of said coefficients consisting of said maximal one of said coefficients, said N^{th} maximal one of said coefficients and any coefficients therebetween, including the relative position of the coefficients relative to one another.

11. The method of claim 10 wherein N is an integer being smaller than said number of said coefficients of said array.

12. The method of claim 10 wherein said finding comprises disregarding an initial polarity of each of said coefficients, said sparse array comprising the N maximal coefficients with their initial polarity.

13. The method of claim 10 further comprising normalizing said N maximal coefficients of said sparse array, the N maximal coefficients ranging between the positive unity and the negative unity.

14. The method of claim 10 wherein said performing includes performing one or more discrete wavelet transforms on said amplitude values of said pulsed signal, said coefficients being wavelet coefficients.

15. The method of claim 10 wherein said pulsed signal is generated by at least one of brain tissue and a heart tissue.

16. A method of stimulating a pulse generating entity, the method comprising:
implanting a probe head within the pulse generating entity, the probe head having a stimulation element and an electrode;
during a given time window, the electrode collecting a corresponding plurality of electrical signals; and
upon associating at least a threshold number of said electrical signals to a common reference pulse shape, stimulating the pulse generating entity using the stimulation element, including generating a stimulation signal within the pulse generating entity.

17. The method of claim 16 wherein said stimulation signal is selected on the basis of the common reference pulse shape of the threshold number of electrical signals.

18. The method of claim 16 wherein said stimulation signal is an optical stimulation signal.

19. The method of claim 16 wherein said stimulation signal is associated with a reaction of said pulse generating entity, the method further comprising conditioning the pulse generating entity to react according to the reaction via said stimulating.

20. The method of claim 16 wherein said pulse generating entity is at least one of brain tissue and heart tissue.
